(19) United States
(12) Patent Application Publication
(10) Pub. No.: US 2025/0082877 A1
(43) Pub. Date: Mar. 13, 2025

(54) RESPIRATORY SUPPORT SYSTEM

(71) Applicant: Fisher & Paykel Healthcare Limited, Auckland (NZ)

(72) Inventors: Cameron John BREWSTER, Auckland (NZ); Edward John EVANS, Auckland (NZ); Arjen David KAT, Auckland (NZ); Bruce Gordon HOLYOAKE, Auckland (NZ); Gareth Thomas MCDERMOTT, Auckland (NZ); Cameron Leslie MATTHEWS, Auckland (NZ); Hannah Maree MATTHEWS, Auckland (NZ); Evan Anthony MAUNDER, Auckland (NZ); Winnie Yong JIANG-FOO, Auckland (NZ)

(21) Appl. No.: 18/722,942

(22) PCT Filed: Dec. 23, 2022
(86) PCT No.: PCT/IB2022/062713

§ 371 Date: Jun. 21, 2024

Related U.S. Application Data
(60) Provisional application No. 63/265,954, filed on Dec. 23, 2021.

(51) Int. Cl. A61M 16/00; A61M 16/08; A61M 16/12; A61M 16/20

(52) U.S. Cl. CPC A61M 16/0066; A61M 16/0003; A61M 16/022; A61M 16/0816; A61M 16/12; A61M 16/208; A61M 2016/0033; A61M 2206/12

(57) ABSTRACT

A device for providing respiratory gases comprises a blower configured to receive a first gas from a first gas flow path and generate a flow of a first gas provided through a first gases outlet of the blower; a second gas flow path configured to receive a flow of a second gas and provide the flow of the second gas through a second gases outlet; and a mixing chamber configured to receive a flow of the first gas from the first gases outlet and a flow of the second gas from the second gases outlet. The received gases are configured to mix in the mixing chamber to form a mixed gas. The received gases are configured to travel in a mixing flow direction in the mixing chamber towards a mixed gases inlet, wherein mixed gas exits the mixing chamber via the mixed gases inlet providing flow to a mixed gas flow path.

This application claims priority from U.S. Application No. 63/265,954 filed on 23 Dec. 2021, the contents of which are to be taken as incorporated herein by this reference.

### TECHNICAL FIELD

The present disclosure relates to devices and systems delivering respiratory support to a patient. It relates particularly, but not exclusively, to a system for providing high flow respiratory support providing a flow of mixed gases to a patient, and to devices for use with the system in the provision of respiratory support.

### BACKGROUND OF INVENTION

Patients with diminished respiratory function or risk of diminished respiratory function can benefit from high flow respiratory support. Patients may lose respiratory function during anaesthesia, or sedation, or more generally during certain medical procedures. Prior to a medical procedure a patient may be pre-oxygenated by a medical professional to provide a reservoir of oxygen saturation, and this pre-oxygenation is generally carried out with a bag and a face mask. Once under general anaesthesia, patients must be intubated to ventilate the patient. In some cases, intubation is often completed in under 60 seconds, but in other cases, particularly if the patient's airway is difficult to traverse (for example, due to cancer, severe injury, obesity or spasm of the neck muscles), intubation may take significantly longer. While pre-oxygenation provides a buffer against declines in oxygen saturation, for long intubation procedures, it is necessary to interrupt the intubation process and reapply the face mask to increase the patient's oxygen saturation to adequate levels. The interruption of the intubation process may happen several times for difficult intubation processes, which is time consuming and can potentially put the patient at risk. After approximately three attempts at intubation the medical procedure will be abandoned.

Other situations where a patient may experience diminished respiratory function that could benefit from delivery of high flow respiratory support include where the patients experience respiratory disorders, as are frequently encountered in Intensive Care Units (ICUs).

The present disclosure provides systems and devices for providing respiratory support, particularly high flow respiratory support.

A reference herein to a patent document or any other matter identified as prior art, is not to be taken as an admission that the document or other matter was known or that the information it contains was part of the common general knowledge as at the priority date of any of the provisional claims.

### SUMMARY OF INVENTION

Viewed from one aspect, the present disclosure provides a device for providing respiratory gases, the device comprising: (a) a blower configured to receive a first gas from a first gas flow path and generate a flow of a first gas provided through a first gases outlet of the blower; (b) a second gas flow path configured to receive a flow of a second gas and provide the flow of the second gas through a second gases outlet; and (c) a mixing chamber configured to receive a flow of the first gas from the first gases outlet and a flow of the second gas from the second gases outlet, the received gases configured to mix in the mixing chamber to form a mixed gas, the received gases configured to travel in a mixing flow direction in the mixing chamber towards a mixed gases inlet, wherein mixed gas exits the mixing chamber via the mixed gases inlet providing flow to a mixed gas flow path.

In some embodiments, the mixing chamber receives the flow of gases from the second gases outlet upstream of the first gases outlet in the mixing flow direction.

In some embodiments, one or both of the first gases outlet and the second gases outlet are arranged to achieve flow of the first gas and/or the second gas substantially tangentially along a wall of the mixing chamber.

In some embodiments, the first gas flow path is an oxygen flow path.

In some embodiments, the first gases outlet and the second gases outlet are arranged relative to the mixing chamber such that the first gases outlet is arranged to direct the first gas entering the mixing chamber away from the second gases outlet.

In some embodiments, the first gases outlet and the second gases outlet are arranged such that the first gas in the first gases outlet is directed in a first flow direction between a direction that is substantially parallel to a second flow direction of the second gas in the second gases outlet, and a direction that is substantially perpendicular to the second flow direction. For example, the first flow direction relative to the second flow direction may be at an angle of about 0° to less than about 90°.

In some embodiments, the first flow direction and the second flow direction are in a common plane. Alternatively/additionally, the mixed flow direction, the first flow direction and the second flow direction may be in a common plane.

In some embodiments, the mixed gases inlet is arranged such that the mixed gas flow in the mixed gas flow path is directed in a mixed flow direction between a direction substantially perpendicular to one or both of the first and second flow directions and a direction anti-parallel to one or both of the first and second flow directions.

In some embodiments, the mixed flow direction is substantially anti-parallel to the second flow direction wherein antiparallel has the conventional meaning of directions that are parallel but moving in opposite directions.

In some embodiments, the mixing chamber is substantially circular in cross section. For example, the mixing chamber may be substantially cylindrical.

In some embodiments, the mixing flow direction in the mixing chamber is around a central shaft. The central shaft may comprise a portion of the blower such as the blower motor assembly where the mixing flow direction is around an axis of the blower motor assembly.

In some embodiments, the mixing chamber is configured such that gases travel in the mixing chamber in a spiral manner. This may include travel in a spiral manner as gases exit the first gases outlet into the mixing chamber.

Alternatively/additionally, this may include travel in a spiral manner around an axis of the blower motor assembly.

In some embodiments, the mixing chamber is comprised of a plurality of adjacent sectors, and wherein the first gases outlet and the second gases outlet are arranged in adjacent sectors of the mixing chamber. The plurality of sectors may comprise four quadrants. In some embodiments, the mixed gases inlet may be arranged in a non-adjacent sector to the first gases outlet. The mixed gases inlet may be provided in a sector that achieves optimal uniformity of flow within the mixed gases flow path.

In some embodiments, arrangement of the second gases outlet permits flow of gases from the mixing chamber into the second gas flow path. The second gases outlet may comprise a lead-in portion. The lead in portion may comprise a taper configured to direct flow of the second gas into the mixing chamber.

In some embodiments, the second gas flow path comprises a flow conditioner at the second gases outlet which is configured to increase resistance to flow of gases from the mixing chamber. The flow conditioner may comprise a plurality of substantially parallel flow channels. In some embodiments, the flow conditioner may have an outlet end that is shaped to be continuous with an internal wall of the mixing chamber. In some embodiments, the flow conditioner is formed integrally with the device. The term “second gases outlet” refers to the oxygen outlet introducing O₂ to the mixing chamber.

In some embodiments, the second gas flow path comprises one or more nozzles configured to provide the flow of the second gas to the mixing chamber through a nozzle diameter which is less than a diameter of the second gas flow path.

In some embodiments, the second gas flow path comprises a non-return valve.

In some embodiments, the second gas flow path comprises a proportional valve.

In some embodiments, the device comprises a first flow sensor for sensing flow rate of gases in the first gas flow path.

In some embodiments, the device comprises a second flow sensor for sensing flow rate of gases in the second gas flow path. The second flow sensor may sense flow rate of gases downstream of the proportional valve when provided.

In some embodiments, the device comprises a third flow sensor for sensing flow rate of the mixed gas in the mixed gas flow path.

In some embodiments, the mixed gas flow path comprises a mixed flow conditioner upstream of the third flow sensor. The mixed flow conditioner may be located at or proximal to the mixed gases inlet. The mixed flow conditioner may be integral with the mixed gases inlet.

In some embodiments, the mixed flow conditioner has an inlet end that is shaped to be continuous with an internal wall of the mixing chamber. The mixed flow conditioner may comprise a plurality of substantially parallel flow channels.

In some embodiments, one or more of the first gas, second gas and mixed gas flows comprise a flow rate of 0 L/min or greater, optionally the mixed gas flow comprises a flow rate of about 20 L/min to about 90 L/min, optionally the mixed gas flow comprises a flow rate of about 40 L/min to about 70 L/min.

In some embodiments, the device comprises a plurality of cooperating components through which bores have been formed which cooperate to define a plurality of gas flow paths, and into which cooperating cavities have been formed to define a cavity for receiving the blower and a mixing chamber.

In some embodiments, the device comprises a pneumatic block having three or more cooperating components, wherein: (a) a first component comprises a first opening defining a first gases inlet, a second opening defining a second gases inlet, and a first cavity for receiving a first part of the blower; (b) a second component comprises three parallel through-bores defining part of each of the first gas flow path, the second gas flow path and the mixed gas flow path, and a third opening defining a device outlet; (c) the third component comprises three parallel through-bores defining part of each of the first gas flow path, the second gas flow path and the mixed gas flow path, and a second cavity for receiving a second part of the blower and defining the mixing chamber; wherein the bores in the second component align with the bores in the third component to define colinear parts of the first gas flow path, second gas flow path and mixed gas flow path. In some embodiments, the device comprises a housing.

Viewed from another aspect, the present disclosure provides a device for providing respiratory gases, the device comprising: (a) a blower configured to generate a flow of a first gas provided through a first gases outlet; (b) a second gas flow path configured to receive a flow of a second gas and provide the flow of the second gas through a second gases outlet; (c) a mixing chamber configured to receive a flow of the first gas from the first gases outlet and a flow of the second gas from the second gases outlet, the received gases configured to mix in the mixing chamber to form a mixed gas, the received gases configured to travel in a mixing flow direction in the mixing chamber towards a mixed gases inlet; wherein the mixed gas exits the mixing chamber via the mixed gases inlet providing flow to a mixed gas flow path; and wherein the mixed gases inlet is positioned in the mixing chamber relative to one or both of the first gases outlet and second gases outlet to achieve optimal uniformity of flow within the mixed gas flow path.

In some embodiments, the first gases outlet is arranged such that flow from the first gases outlet is directed in the mixing chamber away from the mixed gases inlet.

In some embodiments, the second gases outlet is arranged such that flow from the second gases outlet is directed in the mixing chamber away from the mixed gases inlet.

In some embodiments, the first gases outlet and the second gases outlet are arranged such that the first gas in the first gases outlet is directed in a first flow direction between a direction that is substantially parallel to a second flow direction of the second gas in the second gas outlet, and a direction that is substantially perpendicular to the second flow direction. The first flow direction relative to the second flow direction may be at an angle of about 0° to less than about 90°. In some embodiments, the first flow direction and the second flow direction are in a common plane.

In some embodiments, the mixed gases inlet is arranged such that mixed gas flow in the mixed gas flow path is directed in a mixed flow direction between a direction substantially perpendicular to one or both of the first flow direction and the second flow direction, and a direction anti-parallel to one or both of the first flow direction and the second flow direction. The first direction and the second direction may be in a common plane.

In some embodiments, the device comprises a mixed gas flow sensor for sensing flow rate of the mixed gas in the mixed gas flow path. In some embodiments, the mixed gas flow path comprises a mixed flow conditioner upstream of the mixed gas flow sensor. In some embodiments, the mixed flow conditioner is located at the mixed gases inlet. In some embodiments, the mixed flow conditioner has an inlet end that is shaped to be continuous with an internal wall of the mixing chamber. The mixed flow conditioner may comprise a plurality of substantially parallel flow channels. In some embodiments, the mixed flow conditioner may be integral with the mixed gases inlet.

In some embodiments, the mixing chamber is substantially circular in cross section. The mixing chamber may be substantially cylindrical.

In some embodiments, the mixing flow direction in the mixing chamber is around a central shaft. The central shaft may comprise a portion of the blower such as the blower motor assembly where the mixing flow direction is around an axis of the blower motor assembly.

In some embodiments, the mixing chamber is configured such that gases travel in the mixing chamber in a spiral manner.

In some embodiments, the mixing chamber is comprised of a plurality of adjacent sectors, wherein the first gases outlet and the second gases outlet are arranged in adjacent sectors of the mixing chamber. The plurality of sectors may comprise four quadrants. In some embodiments, the mixed gases inlet is provided in a non-adjacent sector to the first gases outlet. The mixed gases inlet may be provided in a sector that achieves optimal uniformity of flow within the mixed gases flow path. In some embodiments, arrangement of the second gases outlet permits flow of gases from the mixing chamber into the second gas flow path.

In some embodiments, one or more of the first gas, second gas and mixed gas flows comprise a flow rate of 0 L/min or greater, optionally the mixed gas flow comprises a flow rate of about 20 L/min to about 90 L/min, optionally the mixed gas flow comprises a flow rate of about 40 L/min to about 70 L/min

Viewed from another aspect of the disclosure, there is provided a device for providing a flow of respiratory gases, the device comprising a pneumatic block assembly comprising a plurality of cooperating block components configured to provide, when assembled: (a) a first through bore defining a first gas flow path; (b) a second through bore defining a second gas flow path; (c) a cavity defining a mixing chamber; and (d) a third through bore defining a mixed gas flow path; wherein the pneumatic block assembly comprises a material having an unoccupied volume comprised of the through bores and the cavities, and wherein the proportion of unoccupied volume attributable to the through bores is greater than the proportion of unoccupied volume attributable to the cavities.

In some embodiments, the cavity is configured to accommodate a blower.

In some embodiments, the unoccupied volume attributable to the through bores is more than about 50%, preferably more than about 60% and optionally about 64% of the unoccupied volume.

In some embodiments, the unoccupied volume attributable to the cavities is about 20% and optionally about 18% of the unoccupied volume.

In some embodiments, the pneumatic block assembly further comprises one or more sensor cavities, and the unoccupied volume attributable to the sensor cavities is about 20% and optionally about 18% of the unoccupied volume.

In some embodiments, the pneumatic block assembly comprises a metal or metal alloy into which the through bores and cavities have been machined or milled.

In other embodiments, the pneumatic block assembly comprises a metal or metal alloy which into which the through bores and cavities have been formed using a mould.

In some embodiments, the pneumatic block assembly comprises one or more thermally conductive materials. In some embodiments, the pneumatic block assembly comprises one or more materials selected from a group comprising metals, metal alloys, ceramics and polymers.

In some embodiments, arrangement of the first gas flow path, the second gas flow path, the mixed gas flow path and the cavities accommodating the blower and defining the mixing chamber within the pneumatic block assembly provide a compact form factor.

In some embodiments, the pneumatic block assembly comprises a plurality of block components, and wherein a first block component provides a mounting surface to which the other block components are configured to be mounted.

In some embodiments, the pneumatic block assembly comprises a mounting element configured to cooperate with a mounting structure to which the device may be mounted during use.

In some embodiments, the device comprises a housing. The mounting element may be provided through the housing.

In some embodiments, the housing contains a ventilating blower configured to ventilate inside the housing. The housing may comprise a baffle configured to direct flow from the ventilating blower over the pneumatic block inside the housing. In some embodiments, the flow from the ventilating blower is separate from the flow of respiratory gases.

In some embodiments, the baffle comprises one or more slots for accommodating electrical components inside the housing. Alternatively/additionally, the baffle may comprise one or more structures to guide air flow from the ventilating blower to an electrical supply connector of the device. Alternatively/additionally, the baffle may comprise one or more features providing structural strength mitigating one or more of sagging, compression or bending of the baffle or part thereof.

In some embodiments, the baffle comprises one or more features splitting air flow from the ventilating blower and optionally, guiding flows over different components of the device such as but not limited to a power distribution component of the device.

In some embodiments, the baffle comprises one or more hollow portions located to engage with one or more protrusions in an inside surface of the housing. The one or more hollow portions may comprise conical sections configured to engage with protrusions comprising screw bosses in the housing.

In some embodiments, the baffle comprises one or more slots configured to cooperate with a protrusion on an internal surface of the housing.

In some embodiments, the baffle is arranged between opposing walls of the housing.

Viewed from another aspect of the disclosure, there is provided a device for providing a flow of respiratory gases, the device comprising: (a) a flow modulator having an inlet and an outlet, the flow modulator configured to provide a flow of gases through the outlet; and (b) a flow conditioner configured to condition the flow of gases from the outlet; wherein the flow conditioner is configured to disperse the flow of the gases entering the flow conditioner and condition the gas exiting the flow conditioner.

The flow modulator may comprise a proportional valve.

In some embodiments, the flow conditioner comprises a first portion configured to receive and disperse the flow of gases. The first portion may comprise a sintered metal filter, preferably a bronze sintered filter. In some embodiments, the first portion comprises a cavity configured to fill with the flow of gases which is disbursed through openings in the filter when pressure within the filter exceeds a filter threshold.

In some embodiments, the flow conditioner comprises a second portion configured to straighten the dispersed gases.

In some embodiments, the first portion comprises an external conical shape having a tip configured to be received in a corresponding recess in the second portion. In some embodiments, the recess comprises a through hole. The tip may be shaped to key or cooperate with the recess in the second portion.

In some embodiments, the second portion comprises a plurality of openings. The openings may have a cross section which is substantially circular. In some embodiments, the openings in the second portion provide a honeycomb structure. In some embodiments, the second portion comprises a plurality of parallel flow channels. In some embodiments, the plurality of flow channels have a length which may be non-uniform between the plurality of flow channels. The plurality of flow channels may be of uniform or non-uniform diameter. The plurality of flow channels may be of uniform or non-uniform cross-sectional shape. In some embodiments, the plurality of flow channels are arranged radially in the second portion.

In some embodiments, the plurality of flow channels are arranged in the second portion such that they are entirely within the bounds of a flow channel downstream of the flow conditioner.

In some embodiments, the flow of gases exit the outlet at a high velocity and/or a cross-sectional area of the flow of gases exiting the outlet is less than a cross-sectional area of a flow path into which it enters.

Viewed from another aspect of the present disclosure, there is provided a device for providing a flow of respiratory gases, the device comprising: an inlet; and an outlet to provide the flow of respiratory gases to a patient, the outlet comprising an outlet connector configured to couple with a delivery connector to provide the flow of respiratory gases to a patient; wherein the outlet connector comprises an outflow end which is configured to releasably receive the delivery connector, the outflow end comprising a plurality of apertures having an opening size smaller than the delivery connector to prevent over insertion of the delivery connector into the device.

In some embodiments, the plurality of apertures are positioned toward a middle portion of the outlet connector. Thus, the plurality of apertures may be arranged closer to a central axis of the outlet connector than they are to a periphery of the outlet connector.

In some embodiments, the outlet connector comprises an inflow end configured to receive the flow of respiratory gases into the outlet connector.

In some embodiments, the outflow end comprises a central opening and the plurality of apertures.

In some embodiments, the outlet connector is configured to provide a plurality of flow paths when coupled with the delivery connector, comprising at least a central flow path between the inflow end and the central opening, and a plurality of outer flow paths between the inflow end and the plurality of apertures. The plurality of outer flow paths may be substantially parallel to the central flow path.

In some embodiments, the central opening is configured to align with a central opening of the delivery connector.

In some embodiments, the outflow end comprises an internal taper configured to guide insertion of the delivery connector.

In some embodiments, the outflow end is configured to form a sealing engagement with the delivery connector.

In some embodiments, the outflow end has a smaller internal cross-section at or near the plurality of apertures or at or near a middle portion of the outlet connector compared to an internal cross-section at or near a terminal end.

In some embodiments, the device comprises a non-return valve between a mixed gas outlet of the device and an inflow end of the outlet connector.

In some embodiments, the device comprises a pneumatic block defining the first gas flow path, the second gas flow path, the mixed gas flow path, and the mixing chamber, and wherein the non-return valve is downstream of a mixed gases outlet of the pneumatic block. In some embodiments, the outlet connector comprises a connector gasket configured to provide a substantially sealing coupling with the pneumatic block.

In some embodiments, the non-return valve is positioned at an angle such that gravitational forces bias the non-return valve to a closed position when the device is upright.

In some embodiments, the outlet connector is oriented to receive the delivery connector at an angle requiring application of a connection force having both vertical and horizontal force vectors. For example, the outlet connector may be oriented at an angle of approximately 60 degrees to vertical requiring a simultaneous lateral and upward connection force to be applied.

### BRIEF DESCRIPTION OF DRAWINGS

The invention will now be described in greater detail with reference to the accompanying drawings in which like features are represented by like numerals. It is to be understood that the embodiments shown are examples only and are not to be taken as limiting the scope of the invention as defined in the provisional claims appended hereto.

FIG. 1 is a schematic diagram of an example of a respiratory system for providing respiratory gases to a patient.

FIG. 2 is a schematic drawing showing components of a system for delivery of respiratory gases according to embodiments of the present disclosure.

FIGS. 3A to 3C are flow diagrams based on computational fluid dynamic models showing flows of gases within a mixing chamber and flow paths of the device according to various embodiments of the disclosure.

FIGS. 4A to 4D are computational fluid dynamics (CFD) diagrams showing undesirable flows of gases within a mixing chamber and flow paths when the blower outlet is arranged in non-preferred sectors of the mixing chamber.

FIGS. 5A to 5C are end, perspective and side views of a flow conditioner utilised in some embodiments of the disclosure.

FIGS. 6A and 6B are front perspective views of a pneumatic block assembled according to embodiments of the disclosure, comprised of a plurality of block components.

FIG. 7A is a transparent front view of the pneumatic block 700 of FIGS. 6A and 6B with a mixed gas conditioner that is integrally formed with the device. FIG. 7B is a transparent perspective front view of the pneumatic block of FIGS. 6A, 6B and 7A modified with a mixed gas conditioner that is not integrally formed with the device.

FIG. 8 is a transparent rear view of the pneumatic block in FIGS. 6A to 7B showing in particular, features of a first component at the back of the pneumatic block.

FIG. 9 is a diagram showing representation of streamlines of air received in the air inlet of a first component of the pneumatic block, and traveling through a bore in the third component into a cavity in the first component that supplies air to the inlet of blower 310.

FIG. 10A is a schematic illustration of an air flow conditioner that may be provided in an air flow path according to embodiments of the disclosure.

FIG. 10B is a schematic illustration of another air flow conditioner that may be provided in an air flow path according to embodiments of the disclosure.

FIGS. 11A and 11B are end and side views respectively, of a mixed gas flow conditioner that is not integrally formed with the device.

FIGS. 12A to 12C are side, end and sectional views, respectively, of an outlet connector according to embodiments of the disclosure.

FIGS. 13A to 13C show an air inlet of the device with removable filter and removable cover, according to embodiments of the disclosure.

FIGS. 14A to 14C show a second air inlet of the device with removable filter and removable cover, according to embodiments of the disclosure.

FIGS. 15A and 15B show flow paths for within a housing of the device showing a path of least resistance and a guided flow path, respectively.

FIGS. 16A and 16B are opposing perspective views of a baffle for guiding flows within the device housing.

FIGS. 17A and 17B show the baffle of FIGS. 16A and 16B arranged to direct flows across a pneumatic block and IEC connector. FIG. 17B shows the arrangement of FIG. 17A, further showing a display screen.

FIG. 18A shows an oxygen flow conditioner having a first portion and a second portion according to an embodiment of the disclosure. FIG. 18B shows the first portion separate from the second portion which is shown in FIG. 18C.

### DETAILED DESCRIPTION

Embodiments of the disclosure are discussed herein by reference to the drawings which are not to scale and are intended merely to assist with explanation of the invention.

In anaesthetic procedures in which high flow respiratory support is provided, high flow gases can contain oxygen (O₂) levels higher than ambient air (21%). In general anaesthesia procedures, 100% O₂ can be delivered to the patient during pre-oxygenation to build an O₂ reservoir (acting as a buffer) in the patient's lungs and blood before anaesthetic induction, and before intubation (while the patient may be apnoeic) to maintain blood O₂ saturation levels or prevent/reduce a decrease in blood O₂ saturation levels.

Flow rates during general anaesthesia procedures can be as high as 70 L/min or in some cases 90 L/min during apnoeic oxygenation. The combination of high flow rates and high O₂ concentration required to deliver the respiratory support in this context require a system that can reliably, accurately and safely control the delivery of high gas flows and/or high O₂ concentration to patients. Embodiments of the present disclosure may provide improvements in one or more of sensor performance, gas mixing and flow conditioning which can in turn give rise to improvements in system and device performance in the provision of high flow respiratory support. With respect to sensors and their measurements, it is desirable to provide gases with a flow profile which is linear, uniform and/or parallel so as to for example improve sensing accuracy and/or consistency. In some embodiments, flow conditioners may be used to condition gas flows to be closer to a desirable flow profile. Alternatively and/or additionally, flow sensors may be used to determine actual flows within the device so that improved flow control and delivery of gases may be achieved.

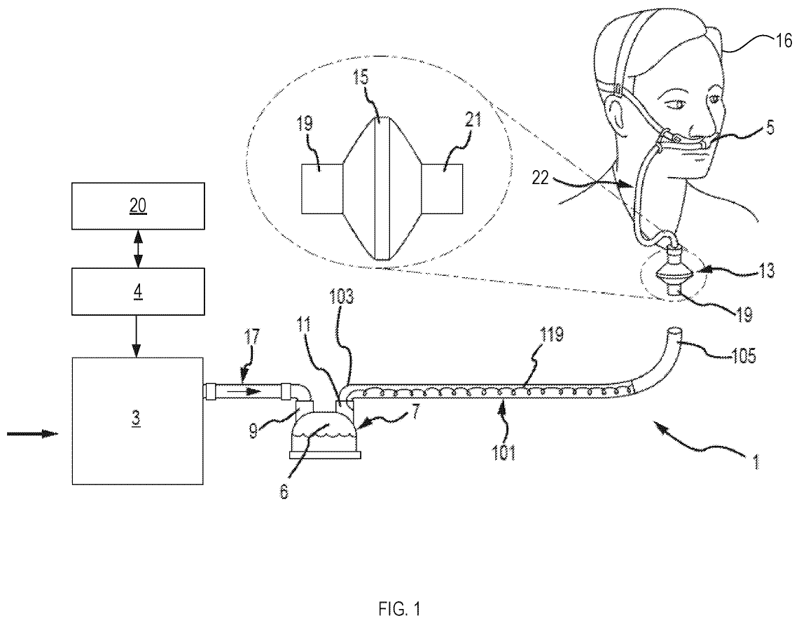

FIG. 1 is a schematic diagram of an example of a respiratory system 1 for providing respiratory gases to a patient. System 1 comprises a flow source 3 such as an in-wall source of O₂, an O₂ tank, a blower, a flow therapy apparatus, or any other source of O₂ or other gas or combination thereof. In some embodiments, the flow source 3 comprises a flow modulator and in some embodiments, the flow modulator comprises a flow generator such as a blower that provides gases flow comprising a mix of air and O₂ at high flow rates as controlled by a controller 4. Respiratory gases from flow source 3 travel via a first conduit 17 and an inlet 9 to a humidification chamber 6 of humidifier 7 where the gases are conditioned to a predetermined temperature and/or humidity as determined by controller 4.

The humidifier 7 is configured to condition the gases to a pre-determined temperature and/or humidity before delivery to the patient. The flow of respiratory gases provided to the patient may be humidified or, in certain implementations, non-humidified. The humidifier 7 may also include a humidification base unit. In an example, the humidification base unit comprises a heating element operable to heat a humidification fluid in the humidification chamber 6, for example via a conductive base in the chamber 6. The first conduit 17 may provide a conduit for delivering dry flow of gases to the humidifier 7. The first conduit 7 may be coupled with the humidification chamber 6 of the humidifier 7 as shown. Alternatively, the humidifier 7 may be a single component (not shown) and exclude the separate humidification chamber 6 and/or base unit. The humidifier 7 may be configured to condition the gas provided by the flow source 3 to a required temperature and/or humidity. The required temperature and/or humidity may be determined according to the respiratory support being delivered and may be selected by the user or operator to be suitable for the respiratory support to be provided.

Humidified and/or warmed respiratory gases exit humidifier 7 via outlet 11 to which is coupled an inspiratory conduit 101 which delivers the conditioned respiratory gases to the patient 16 via patient interface 5. Typically for the delivery of high flows using system 1, the patient interface is a non-sealing interface such as a non-sealing nasal cannula. In other embodiments, patient interface 5 may be a sealing interface, such as a nasal mask, full face mask or nasal pillows. In some embodiments, humidified gases in the inspiratory conduit 101 may be heated by a heating element 119 provided to or in the inspiratory conduit. In some embodiments, an optional filter 13 may be provided to filter the gases provided to the patient 16. Optional filter 13 may also be provided to prevent contamination for example, of the humidification chamber and the inspiratory conduit in the event of reverse flow from the patient. The optional filter 13 comprises an inlet end 19 configured to couple with the inspiratory conduit 101 at coupling 105. Gases entering inlet end 19 are passed through filter 15 and exit filter outlet end 21 for delivery to the patient interface 5 via filtered gas conduit 22.

Controller 4 includes an input-output interface (I/O interface) 20 which is configured to receive user inputs according to the respiratory support to be provided to the patient, and which may in turn communicate to the user by a screen or audible means such as a speaker, when one or more alarm conditions have been met. The controller 4 comprises or is in operative communication with one or more memory components configured to cause the processor to execute instructions for controlling the flow of respiratory gases according to one or more protocols stored in the memory.

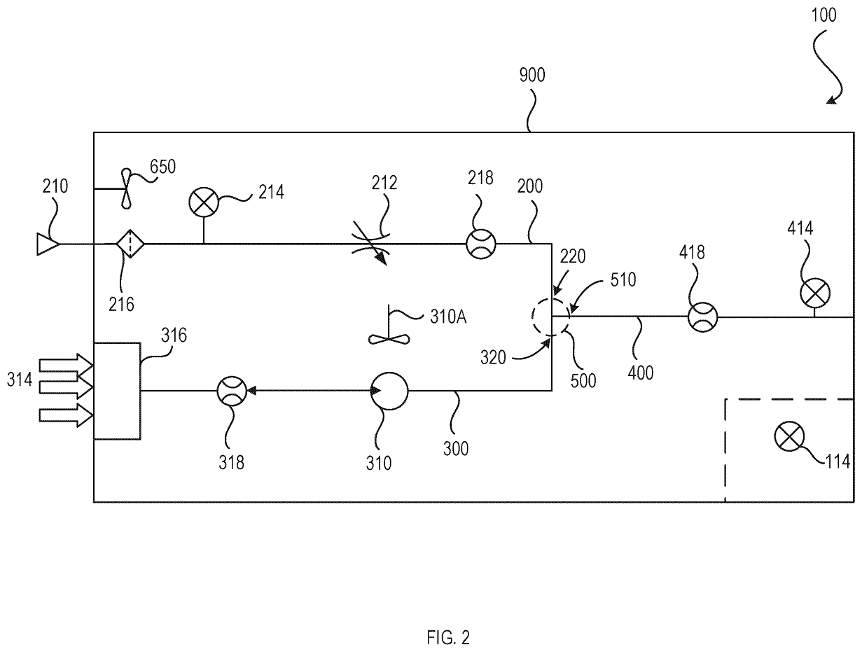

In some embodiments, a user provides to the I/O interface 20 the respiratory support requirements such as gases composition (e.g. O₂ concentration), flow rate and/or pressure to be delivered to the patient's airways. The controller 4 then computes the control signals required for operation of components of the system to deliver that flow rate, pressure and/or O₂ concentration by controlling the flow source 3, and/or various components within the system (such as proportional valve 212—see FIG. 2) to modulate the flow of gases. Controller 4 may receive a plurality of sensor inputs that are used by the controller to determine the control signals as will be described herein. In some embodiments, the I/O interface 20 displays one or more parameters of the system, for example flow rate of gases, pressure (e.g. patient, system, etc), temperature, gas concentration e.g. O₂ concentration etc, which may be received from one or more input, for example controller 4 and sensors.

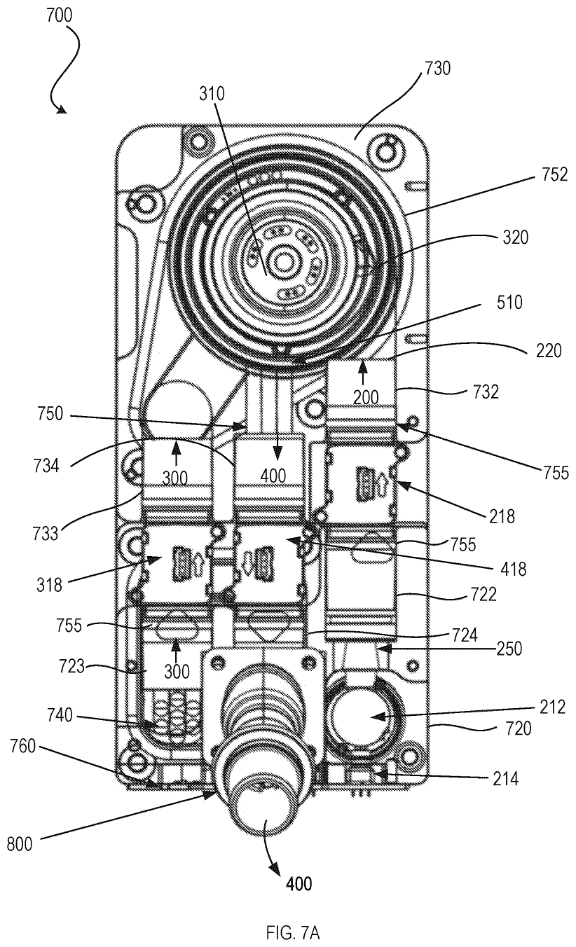

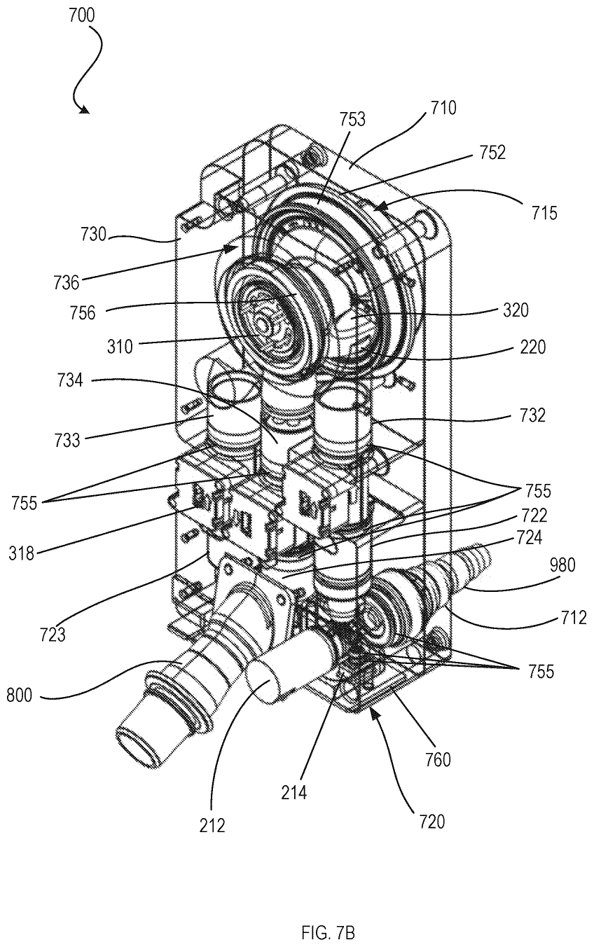

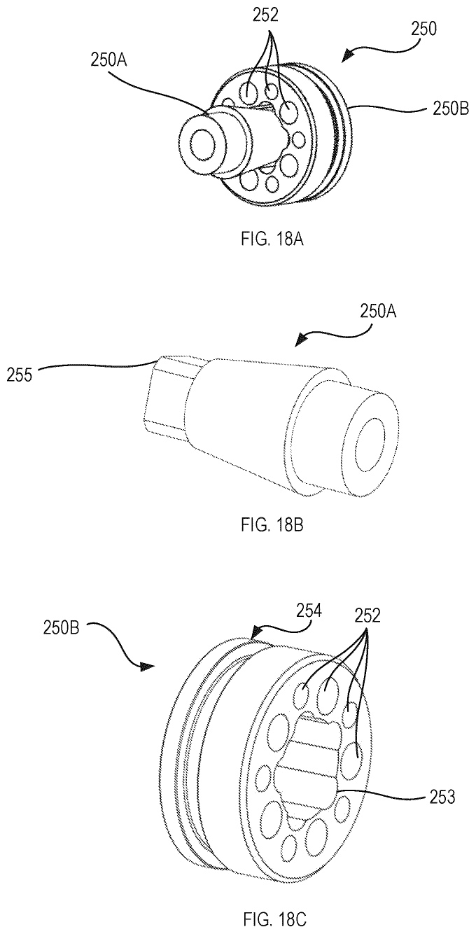

In some embodiments, the present disclosure provides a device comprised of mechanical, electrical and electronic components arranged to provide a flow of gases that delivers the required respiratory support safely, and/or efficiently. FIG. 2 is a schematic drawing showing components of a device 100 for providing respiratory gases according to embodiments of the present disclosure. The device 100 comprises an O₂ flow path 200, air flow path 300 and a mixed gases flow path 400. O₂ flow path 200 is in fluid communication with an O₂ supply 210 which may be a high pressure O₂ supply. Device 100 is representative of flow source 3 of FIG. 1. In an example, the flow source 3 may also include O₂ supply 210. The flow rate of gases in the O₂ flow path 200 is controlled by a proportional valve 212 which is operatively coupled with controller 4. Air flow path 300 has a blower 310 which draws ambient air from air intake 314. The flow rate of gases in the air flow path 300 is controlled by the blower 310 which is operatively coupled with controller 4. Air from air intake 314 may be filtered by air filter 316 to remove particulates. Similarly, a filter 216 may be provided to filter small particles (e.g. <100 um) from the O₂ supply 210. This filter may be placed inside an O₂ connector that is coupled with the inlet of the O₂ supply 210. O₂ is mixed with air downstream of blower 310 to form a mixed gas flow in mixed gas flow path 400 which is delivered as a flow of respiratory gases to the patient. A flow conditioner such as flow conditioner 250 shown in FIGS. 7A and 18A-C may be provided downstream of the proportional valve 212 and upstream of O₂ flow sensor 218.

Various sensors are also provided such as O₂ pressure sensor 214 which is configured to sense O₂ pressure in the O₂ flow path (e.g. to determine that there is a flow of O₂ entering the O₂ flow path), O₂ flow sensor 218 which is configured to sense flow rate in the O₂ flow path 200, air flow sensor 318 which is configured to sense flow rate in the air flow path 300, and mixed gas flow sensor 418 which is configured to sense flow rate in the mixed gas flow path 400 which is delivered to the patient. Additionally, one or more gauge pressure sensors 414 may be provided in the mixed gas flow path 400 and one or more ambient pressure sensors 114 may be provided to sense ambient air pressure. The gauge pressure sensor(s) 414 may take reference from one or more ambient pressure sensors 114 to measure the pressure in the mixed gas flow path 400. The flow rate and pressure of mixed gases in the mixed gas flow path 400 may be controlled by operation of the blower 310 and/or proportional valve 212.

Mixing of gases from the O₂ flow path 200 and air flow path 300 occurs in a mixing chamber 500 shown in broken lines in FIG. 2, where gases from the O₂ flow path 200 enter the mixing chamber through an O₂ outlet 220 and gases from the air flow path 300 enter the mixing chamber through an air outlet 320. As will become apparent below, owing to the physical arrangement of the blower 310 in the air flow path 300 in some preferred embodiments, air outlet 320 may in some embodiments be referred to as blower outlet 320. The mixing chamber 500 may be circular in cross section, such as e.g. cylindrical or spheroid, or may have an oval or obround cross section such that flow of gases in the mixing chamber may be minimally interrupted or disturbed by corners or other internal features of the mixing chamber. Mixing chamber 500 has a mixed gases inlet 510 through which mixed gases from the mixing chamber flow to the mixed gases flow path 400.

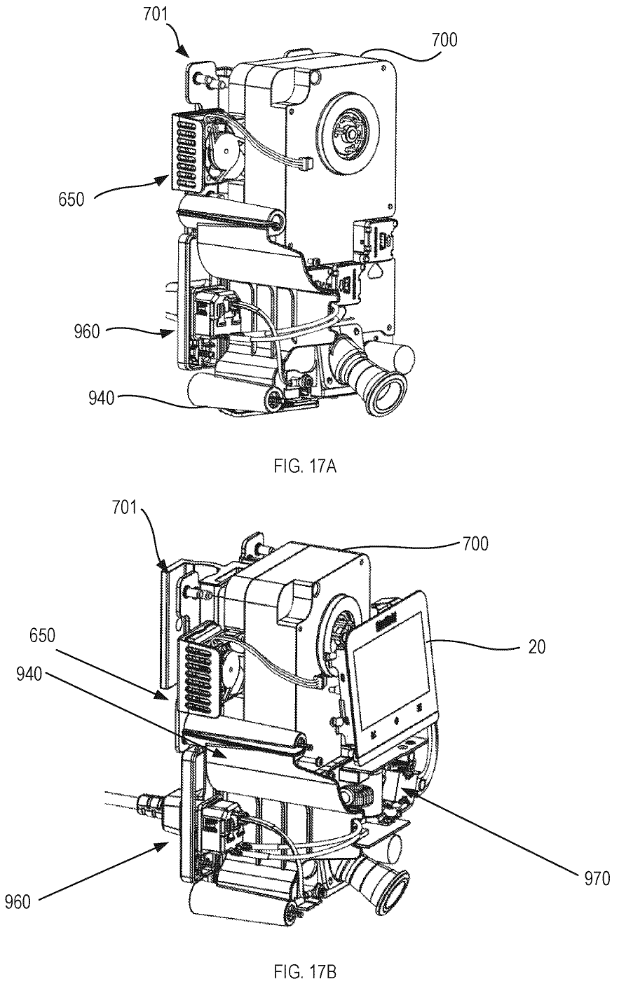

Device 100 may be provided in a housing 900 which may further comprise a ventilating blower 650 to improve safe operation of the device as will be described in further detail below. Not shown in the schematic layout of FIG. 2 is the electrical input to the device 100 which supplies each of the electrically powered components. Electrical input to the device 100 is by an IEC connector. The IEC connector may be connected to an IEC retainer 960 (See FIGS. 17A and 17B). Both the IEC connector and the IEC retainer are of the type known to persons skilled in the art.

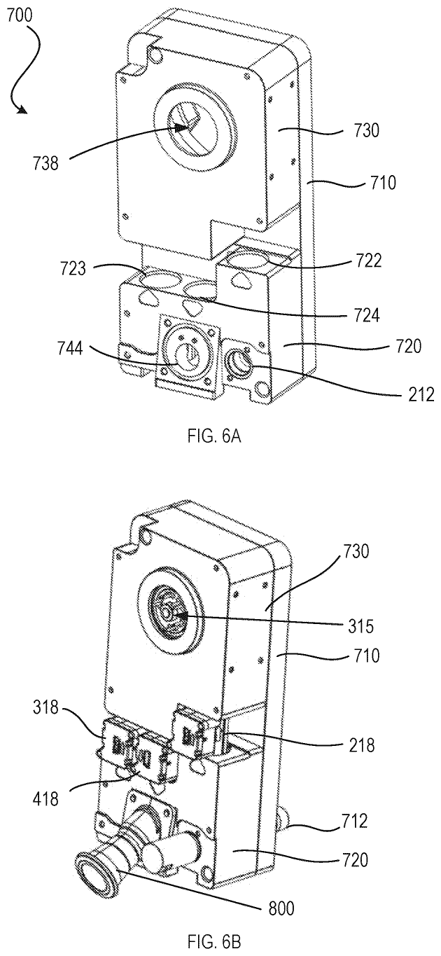

Components of the device 100 that direct flow of gases within the device are provided in a substantially sealed pneumatic block 700 (e.g. as shown in FIGS. 6A & 6B) as will be described in further detail below. As shown in the schematic drawing of FIG. 2, the O₂ flow path 200 and the air flow path 300 are represented as parallel flow paths. Relevantly, when the schematic representation of device 100 of FIG. 2 is manifested in a mechanical device 100, it may also be desirable in some embodiments, for the O₂ flow path 200 and the mixed gas flow path 400 to be arranged in the device such that at least part of those flow paths are arranged in a parallel configuration, as will be described below.

In some embodiments, the O₂ flow path 200 and the air flow path 300 and particularly the O₂ outlet 220 the air outlet 320 are arranged relative to one another to reduce or prevent flow from entering the O₂ path in a reverse flow direction (i.e. against the direction of bulk flow of O₂ entering the O₂ flow path from O₂ source 210). It is desirable to reduce or prevent reverse flow from entering the O₂ path as this could affect the accuracy of sensing by O₂ flow sensor 218. Inaccurate sensing of O₂ flow can affect the control signal provided to proportional valve 212 which can in turn have negative consequences on the accuracy and safety of respiratory gases provided to the patient.

For example in the embodiment of FIG. 2 where the O₂ concentration in a flow delivered to the patient is calculated based on the flow sensors 218, 318 and the concentration of O₂ in the O₂ flow path (e.g. 100% O₂) and O₂ in the air flow path (which is at 21%), when a user provides an input to I/O interface 20 to supply a delivered gas flow with a 21% fraction of O₂ to the patient, there should be zero to low flow in the O₂ flow path since 21% O₂ is largely representative of ambient air. However if an undesirable flow occurs in the O₂ flow path 200, the flow sensor 218 may register a negative or positive flow reading which could cause controller 4 to register that there is less or more than 21% O₂ in the delivered gas flow. This could cause the controller to, in the case of less than 21% O₂ in the delivered flow, open proportional valve 212 to allow more O₂ to flow to the patient, or in the case of more than 21% O₂ in the delivered flow, to close the proportional valve 212 further or register an error if the proportional valve 212 cannot be closed further. Additional O₂ may not be desirable in some applications, for example when O₂ supply is limited. Embodiments of the present disclosure seek to mitigate such issues by careful placement of the blower outlet 320 relative to the O₂ outlet 220, and may utilise a plurality of flow sensors, such as flow sensors 218, 318, 418 to accurately monitor and control the fraction of O₂ in the mixed gas flow path 400. The risk of this error occurring could also be reduced by adding an O₂ concentration sensor into one or more flow paths, for example the O₂ flow path 200 or the mixed gas flow path 300.

In some embodiments, it may be desirable to deliver respiratory gases containing 100% O₂. In such a scenario, the user would enter this O₂ concentration set point into I/O interface 20. The controller 4 then controls proportional valve 212 to open sufficiently (e.g. by increasing supply current) to allow enough O₂ into the flow of respiratory gases to meet the O₂ concentration set point. This may create a pressure downstream of the blower 310, such that only O₂ is delivered to the patient; the blower 310 will still be operating to control flow and pressure however the proportional valve 212 acts to prevent dilution of the O₂ by air from air delivery circuit. In other words, in an embodiment where the device is set to output 100% FiO₂, (wherein FiO₂ is the fraction of oxygen supplied to the patient) the controller 4 adjusts both the amount of opening of the proportional valve 212 and the speed of the blower 310 to meet the flow rate requirement set by the user. The blower 310 maintains a pressure to limit O₂ exiting through the air inlet. For example, if the set points are at 70 LPM and 100% FiO₂ and the O₂ flow sensor records 72 LPM, the speed of blower 310 and size of proportional valve 212 opening may be reduced. Thus, the blower 310 still controls the flow to the patient.

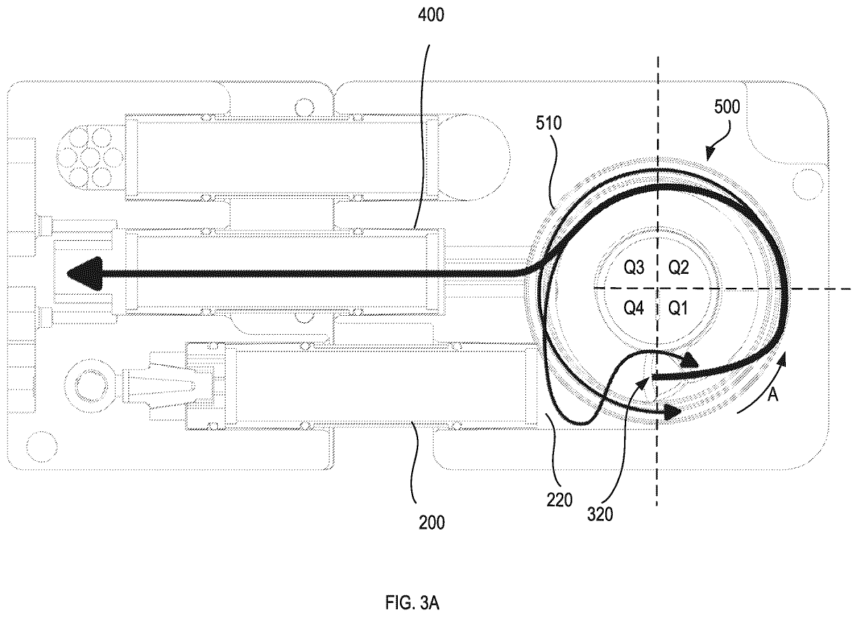

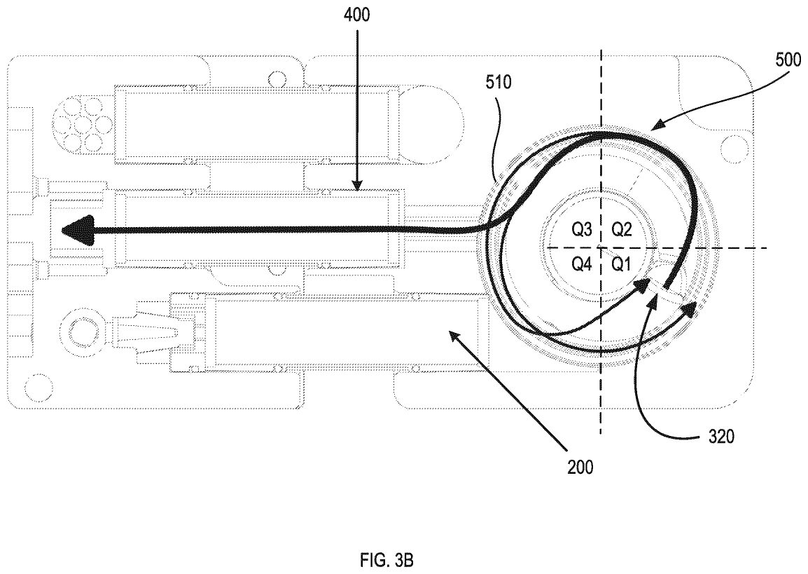

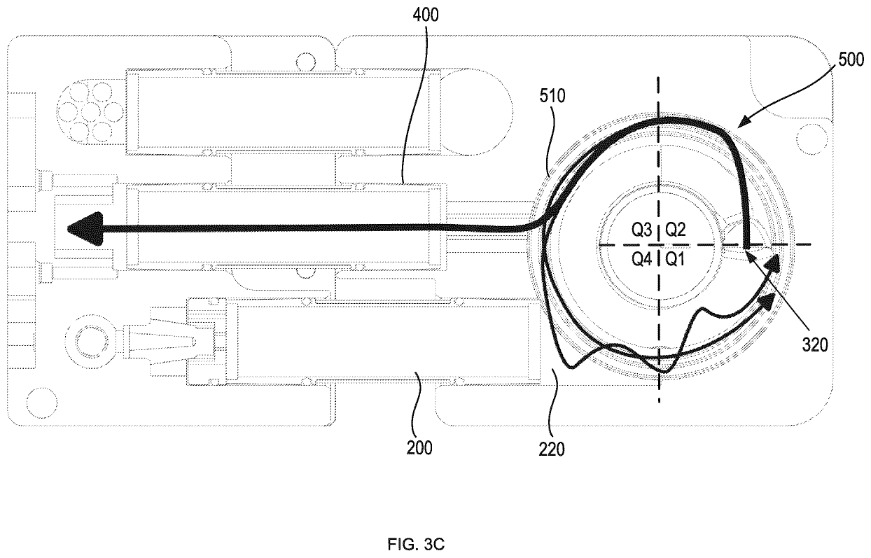

In some embodiments, the mixing chamber 500 receives the flow of gases from the O₂ outlet 220 upstream of the blower outlet 320 in a mixing flow direction within the mixing chamber 500. In some embodiments, the flow direction within the mixing chamber 500 may be represented by direction A in FIG. 3A. In some embodiments, one or both of the blower outlet 320 and the O₂ outlet 220 are arranged to achieve flow of air and O₂ substantially tangentially along a wall of the mixing chamber 500 so as to avoid a flow or backflow of air from the air flow path 300 directly entering the O₂ flow path 200. Alternatively/additionally, the blower outlet 320 and the O₂ outlet 220 may be arranged relative to the mixing chamber such that the blower outlet 320 is arranged to direct air entering the mixing chamber 500 away from the O₂ outlet 220. This means air entering the mixing chamber 500 enters the flow of gases within the mixing chamber such that it travels tangentially within the mixing chamber, away from the O₂ outlet 220 when the O₂ outlet and the air outlet 320 are in a common plane together with the flow of gases in the mixing chamber 500. However it is also contemplated that in some embodiments these outlets may not be provided in a common plane and in such embodiments, the blower outlet 320 and O₂ outlet 220 may be arranged such that air flow into the mixing chamber 500 flows in a direction away from the O₂ outlet 220, in three dimensions.

In some embodiments, the blower outlet 320 and the O₂ outlet 220 are arranged such that the air from the blower outlet is directed in a first flow direction and O₂ from the O₂ outlet 220 is directed in a second flow direction, where the first flow direction is between a direction that is substantially parallel to a second flow direction, and a direction that is substantially perpendicular to the second flow direction. Ideally the first flow direction and the second flow direction are in a common plane, although that need not be the case. These flow directions may be explained by reference to FIGS. 3A to 3C which are flow diagrams simulating flows of gases in the device 100 according to various relative placements of the blower outlet 320 and the O₂ outlet 220. In some embodiments, the blower outlet 320 is arranged downstream of the O₂ outlet 220. For ease of explaining the various locations of the blower outlet 320 and the O₂ outlet 220 with reference to the mixing chamber 500, the chamber has been divided into sectors referred to as quadrants, with a first quadrant represented as Q1, a second quadrant represented as Q2, a third quadrant represented as Q3, and a fourth quadrant represented as Q4. It is to be understood there could be more than 4 sectors however for ease of explanation, the sectors are represented by four quadrants in the present disclosure.

In the flow diagram of FIG. 3A, blower outlet 320 is arranged such that flow exiting the blower outlet enters Q1 such that the first flow direction relative to the second flow direction is at an angle of 0°, that is the first flow direction and second flow direction are substantially parallel. The flow diagram shows that in general, flows from blower outlet 320 enter the mixing chamber 500 and travel annularly through Q1, Q2 and Q3 before most of the flow exits the mixing chamber through mixed gas inlet 510. Flow that remains in the mixing chamber 500 may be recirculated and preferably does not enter the O₂ outlet 220.

In the flow diagram of FIG. 3B, blower outlet 320 is arranged such that flow exiting the blower outlet enters Q1 such that the first flow direction relative to the second flow direction is at an angle of approximately 60°. In the flow diagram of FIG. 3C, blower outlet 320 is arranged in Q1 such that the first flow direction relative to the second flow direction is at an angle of approximately 90°. In both FIGS. 3B and 3C, the flow diagrams show flows from the blower outlet 320 enter the mixing chamber 500 and travel annularly through the remainder of Q1, Q2 and Q3 and the remainder of Q2 and Q3 respectively, before most of the flow exits the mixing chamber through mixed gas inlet 510.

Uniformity of flow within the mixed gas flow path 400 is also important for accurate flow measurements. Placement of the blower outlet 320 relative to the mixed gas inlet 510 can affect the behaviour of flow in the mixed gas flow path 400 as will now be explained.

In FIGS. 3A to 3C, with the blower outlet 320 arranged such that flow enters Q1 of a circular mixing chamber, flow that enters the mixed gas flow path 400 through mixed gas inlet 510 has a level of uniformity that enables flow sensor 418 to produce values that more accurately represent actual flows in the mixed gas flow path than would be the case if the flows in the mixed gas flow path were less uniform. Ideally, the mixed gas flow path 400 is arranged such that the mixed gas flow is directed in a mixed flow direction, where the mixed flow direction is between (and including) a direction that is substantially perpendicular to one or both of the first and second flow directions and a direction anti-parallel to one or both of the first and second flow directions. Preferably, the mixed flow direction, the first flow direction and the second flow direction are in a common plane although that need not be the case.

In the flow diagrams of FIGS. 3A to 3C, the mixed flow direction is substantially anti-parallel to the second flow direction, wherein antiparallel has the conventional meaning of directions that are parallel but moving in opposite directions.

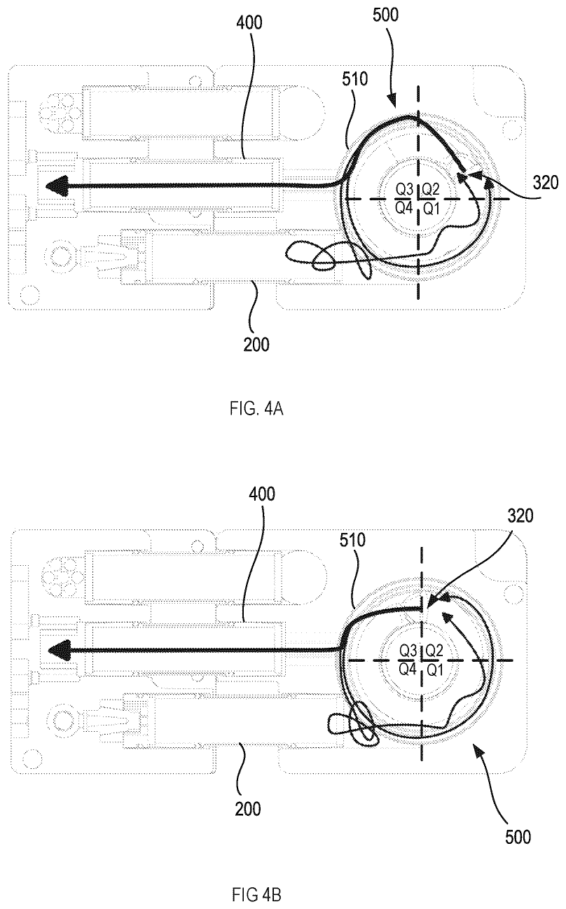

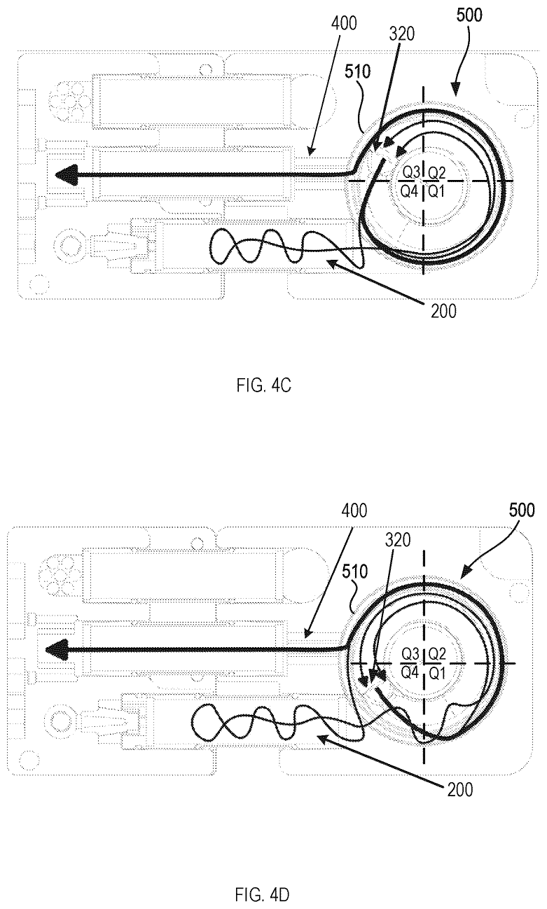

FIGS. 4A to 4D are flow diagrams simulating flows of gases in the device 100 according to various placements of the blower outlet 320 relative to the mixed gas inlet 510 that simulate undesirable characteristics. In FIG. 4A, blower outlet 320 is arranged such that flow exiting the blower outlet enters Q2 to provide a first flow direction at an angle of approximately 120° to the second flow direction. Some of the flow from blower outlet 320 exits mixing chamber 500 via the mixed gas inlet 510 while some flow recirculates in the mixing chamber. The recirculating flow may betravelling at higher velocities compared to the flow velocities simulated in FIGS. 3A to 3C, such that as recirculating flow hits the walls of the mixing chamber 500 it could swirl, and some of the swirling flow enters the O₂ flow path 200 creating undesirable flow within the oxygen flow path. This could cause errors in the detection of bulk gas flow within O₂ flow path 200.

In FIG. 4B, blower outlet 320 is arranged such that flow exiting the blower outlet enters Q3 to provide a first flow direction at an angle of approximately 180° to the second flow direction. Most of the air flow from blower outlet 320 exits mixing chamber 500 via the mixed gas inlet 510. However, flow uniformity within the mixed gas flow path 400 is compromised because flow exiting blower 310 is of high velocity and there is insufficient flow path length for the flow to develop into a desirable flow profile for accurate flow rate sensing. This can have a negative impact on the accuracy of flow sensor 418. In some embodiments, a flow conditioner in the mixed gas flow path 400, for example at the mixed gas inlet 510 may be provided as described in relation to FIGS. 11A and 11B however this may be ineffective in achieving the desired flow profile in some instances where gases flowing from the blower outlet 320 are at high velocities. Hence the blower outlet 320 may be arranged relative to the mixed gas inlet 510 to improve the profile of flows in the mixed gas flow path 400 and reduce risk of flow rate errors being generated by mixed gas flow sensor 418.

In FIG. 4C, blower outlet 320 is arranged in Q3 and creates significant undesirable flows in O₂ flow path 200. This may be attributable to high velocity gases with an undesirable flow profile from the blower entering the mixing chamber 500 causing some of the flow, including recirculating flow, to hit the edges of the O₂ flow outlet 320 which induces a spinning motion in the flow that gives rise to flow entering the O₂ flow path 200 from the mixing chamber. Flow uniformity within mixed gas flow path 400 is also negatively affected which may affect accuracy of operation of mixed gas flow sensor 418. Both of these impacts are undesirable.

In FIG. 4D, blower outlet 320 is arranged such that flow exiting the blower outlet enters Q4. Owing to the high energy of gases exiting the blower, the flow from blower outlet 320 interferes with gases in mixing chamber 500 including those that are in close proximity to the O₂ outlet 200. This also creates undesirable flows in O₂ flow path 200. Flow uniformity within mixed gas flow path 400 is also negatively impacted.

In some embodiments, such as those represented in the flow diagrams of FIGS. 3A to 3C, the flow direction in the mixing chamber 500 may be around a central shaft which may comprise part of the blower 310 such as the blower motor assembly (which comprises a motor and may comprise a motor housing) where the flow direction in the mixing chamber 500 may be around an axis of the blower motor assembly. The axis of the blower motor assembly may be an axis through the length of the blower motor assembly. The axis of the blower motor assembly may also be the axis through the length of the blower. Ideally, the mixing chamber 500 receiving the flow of gases from blower 310 is configured such that gases received in the mixing chamber travel in a spiral manner as this may in some embodiments aid in mixing of gases in the mixing chamber.

As will be apparent from the flow diagrams in FIGS. 3A to 3C and the undesirable flow characteristics in the flow diagrams of FIGS. 4A to 4D, it may be desirable for the mixed gases inlet to be provided in a sector that achieves optimal uniformity of flow within the mixed gases flow path. This may in some embodiments involve arranging the mixed gases inlet 510 in a non-adjacent sector of the mixing chamber 500 to the sector containing the blower outlet 320. In some embodiments, the O₂ outlet 220 may be arranged such that undesirable flow of gases from the mixing chamber 500 into the O₂ flow path 200 is not precluded. That is, there is no one way valve or other flow controlling feature within the O₂ flow path that prevents flow from the flow chamber 500 into the O₂ flow path 200. Rather, the likelihood of such undesirable flows occurring is minimised by suitably locating the blower outlet 320 relative to the O₂ outlet 220 as described in the foregoing, thereby avoiding undesirable flows in the O₂ flow path 200. Alternatively/additionally, one or more baffles may be provided to guide flow from blower outlet 320 away from the O₂ outlet 220. This may have a further benefit of guiding O₂ around the curvature of the mixing chamber 500, improving mixing and minimising the likelihood of O₂ short circuiting from the O₂ outlet 220 to the mixed gas inlet 420. In some embodiments, however it may be desirable to provide a non-return valve in the O₂ flow path 200 to eliminate the risk of phantom flows. In some embodiments, a non-return valve may be located near the O₂ outlet 220. Therefore, beneficially, one or both of the first gases outlet and the second gases outlet may be arranged to achieve a flow of the first gas and/or the second gas substantially tangentially along a wall of the mixing chamber. In some embodiments the blower outlet 320 and O₂ outlet 220are in substantially the same plane. In some embodiments the blower and/or the oxygen outlet 320,220 are in substantially the same plane as the mixing chamber. In some embodiments the mixed gas inlet 510 is in substantially the same plane as the mixing chamber.

Benchtop testing has shown that when the blower outlet 320 is arranged such that the first flow direction relative to the second flow direction is at an angle of 0° to 90°, undesirable flow in the O₂ flow path is negligible and the gas flow rate measured by the flow sensor 418 in the mixed gas flow path and the flow sensor 318 in the air flow path is substantially accurate and representative of the true gas flow rate. For this benchtop test, the true gas flow rate is the flow rate measured by a reference flow sensor of higher accuracy compared to flow sensors 218, 318 and 418 placed in fluid communication with the outlet 744 of the device. The testing has also shown that the blower outlet 320 arranged at 135°, 180° and 270° produces undesirable flow in the O₂ flow path which can affect the O₂ flow sensor accuracy. The sensing accuracy of the flow sensor 418 in the mixed gas flow path or the flow sensor 318 in the air flow path or both were worse at angles of 135°, 180° and 270° compared to positions between 0° to 90°.

In some embodiments, the O₂ outlet 220 comprises a lead in portion. The lead in portion may comprise a taper configured to direct the flow from the O₂ flow path 200 into the mixing chamber 500. The taper may be provided on a portion of an internal wall of a bore or conduit defining the O₂ flow path 200, or the entirety of the internal wall may be tapered so as to form a nozzle. In other embodiments, O₂ flow path 200 may comprise a plurality of nozzles configured to provide a nozzle diameter which is less than a diameter of the O₂ path. In some embodiments, the lead in portion or the nozzles may provide a resistance to flows from the mixing chamber 500 that minimises the likelihood of phantom flows entering the O₂ flow path 200 in the reverse flow direction.

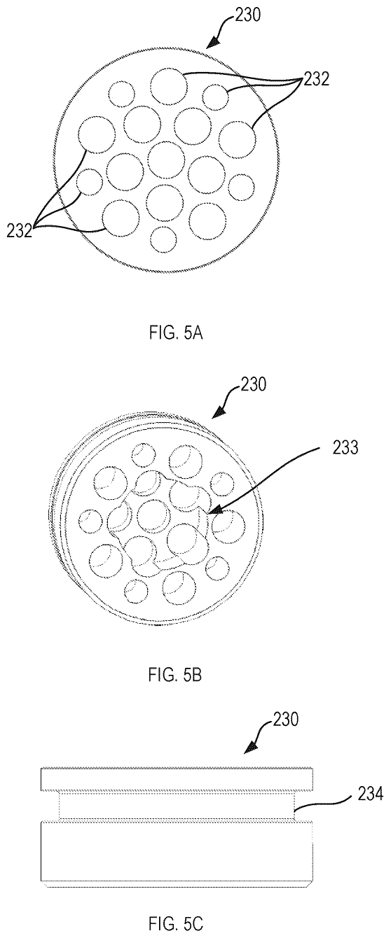

In some embodiments, the O₂ flow path 200 comprises a flow conditioner at the O₂ outlet 220 which is configured to increase resistance to flow of gases from the mixing chamber. An example of a suitable flow conditioner 230 is described in relation to FIGS. 5A and 5B. The flow conditioner may be formed integrally with the device such as a conduit or bore forming the O₂ flow path 200 however in embodiments wherein the flow conditioner 230 is provided as a separate component the flow conditioner 230 may form a sealing join with the bore or conduit by use of an O-ring as will be described in further detail in relation to the pneumatic block in FIGS. 6A to 8.

In some embodiments the flow conditioner 230 comprises a plurality of substantially parallel flow channels which may be circular, oval, elliptical, hexagonal or other cross sectional shape, or a combination of these. FIGS. 5A to 5C show an example of a flow conditioner 230 comprising a plurality of flow channels 232 having circular cross section. It is to be noted that the flow channels need not share a common cross sectional dimension of diameter (for round flow channels), as shown in FIG. 5A. In some embodiments, flow conditioner 230 has an outlet end that is shaped to be continuous with an internal wall of the mixing chamber 500 so as to avoid or minimally disrupt flows within the mixing chamber.

In some embodiments, the device 100 is produced from a plurality of cooperating components through which bores and cavities have been formed. The bores cooperate to define a plurality of gas flow paths and the cavities cooperate to define a space into which the blower may be received while also forming the mixing chamber. In some embodiments, the cooperating components comprise a pneumatic block having a plurality of cooperating components.

In some embodiments, the flow path schematic of FIG. 2 may be materialized within a pneumatic block 700 comprised of three pneumatic block components as illustrated in FIGS. 6A to 8.

When assembled, the pneumatic block 700 provides a substantially sealed system into which a cavity is formed to house the blower 310. The cavity also defines a mixing chamber 500. The assembled pneumatic block 700 contains flow paths for O₂, air and mixed gases. Ideally, the block is designed with specific fluid entrances and exits that control flow of gases within the block although it is to be understood that the flow paths described herein need not be materialized in a pneumatic block; in some embodiments the flow paths or parts thereof may be materialised by conduits and connectors arranged to provide the functionality of the respiratory devices as described herein, as would be understood by one of skill in the art. However, provision of aspects of the respiratory system using a pneumatic block comprised of cooperating block components as described herein may provide several advantages which, in addition to control of flow of gases, may also include a compact form factor. By maintaining good control of gas flows within the device, the safety of the device may be improved.

In some embodiments, the pneumatic block comprises three (or more) cooperating components illustrated in FIGS. 6 to 8 as first block 710, second block 720 and third block 730. Ideally the block components are machined such as milling, drilling, or using other machining techniques to form one or more cavities to accommodate the blower and to define the mixing chamber 500, and to form the bores which cooperate to define the O₂ flow path 200, the air flow path 300 and the mixed gas flow path 400. In some embodiments, the block components are also fabricated to accommodate one or more sensors and flow conditioners as will be explained herein. While the pneumatic block components 710, 720 and 730 are described as being milled to form the requisite cavities and bores, it is to be understood that where these block components are metallic, other metal fabrication techniques may be adopted. It is to be understood, however, that the material structure of the components need not be metallic, and one or more of the block components may comprise polymeric, ceramic or other materials or combinations of materials that may be manufactured using injection moulding or other fabrication techniques to perform the function required of the pneumatic block components.

In some embodiments, cavities comprise open channels or recesses that may be configured to cooperate with a corresponding cavity in an opposing block component (e.g. the first block component 710 and the third block component 730) to define a space for receiving the blower, and which may also define the mixing chamber 500. In contrast, through bores may be regarded as closed tunnels extending through a block component having a single entrance and exit, where the tunnel defines a flow path for gas within the device. Since a through bore is a tunnel formed within the block component, there is no place within the tunnel where gases can leak. In some embodiments, it may be desirable that the unoccupied volume of the block attributable to the through bores may be more than about 50%, preferably more than about 60% and optionally about 64% of the unoccupied volume. In some embodiments, the unoccupied volume attributable to the cavities may be about 20% and optionally about 18% of the unoccupied volume. Since the interface between block components containing cooperating cavities presents an opportunity for gas leakage, in some embodiments, it may be desirable to provide a greater proportion of tunnels than cavities in the pneumatic block to lower the likelihood of gas leaks occurring. In some embodiments, the pneumatic block assembly 700 further comprises one or more sensor cavities, and the unoccupied volume attributable to the sensor cavities may be about 20% and optionally about 18% of the unoccupied volume.

In some embodiments, it may be desirable for one or more of the block components 710, 720, 730 to be manufactured from a metal or metal alloy into which the through bores and cavities may be machined or milled, or formed using a moulding process. In some embodiments, it may be desirable for one or more of the block components 710, 720, 730 to be manufactured from a thermally absorptive or conductive material. In some embodiments, the first component 710 provides a mounting surface to which the other block components may be configured to be mounted or attached. Thus, first component 710 may be regarded as providing a substantially rigid back plate.

Since the first component 710 functions as a mounting plate for the other components, it may be desirable that the first component is manufactured from a material with high strength such as aluminium, stainless steel or a high strength polymer. In some embodiments, materials of one or more of the pneumatic block components may be selected so as to reduce the risk of fire and/or minimize effects of fire during operation. Thus, one or more components of the pneumatic block may be manufactured either entirely or in part from e.g. brass and/or stainless steel and/or Aluminium alloy and/or anodized Aluminium alloy (Al alloy). It is to be understood that other materials, similar in their properties to the examples above, may be used.

FIG. 6A is a perspective illustration of a pneumatic block 700 according to embodiments of the disclosure, comprised of three block components 710, 720, 730. Through bores 722, 723, 724 formed in second component 720 define part of the oxygen, air and mixed gas flow paths respectively. These bores run vertically in the embodiment illustrated and may be substantially parallel. Second component 720 also provides a mixed gas outlet 744. Third block component 730 provides an opening 738 for receiving part of the blower 310 which is contained within the space defined by the cooperating cavities in the third component 730 and the first component 710.

FIG. 6B shows the pneumatic block of FIG. 6A showing additional components including O₂ flow sensor 218, air flow sensor 318 and mixed gas flow sensor 418 in addition to part of the blower motor assembly 315 of blower 310 protruding through opening 738. Fluid flow rate sensors may make mass flow measurements using a thermal measurement principal or using other techniques. Due to the compact arrangement of the pneumatic block and its various features, fluid entering the flow sensors may have an undesirable flow profile for flow rate sensing (e.g. non-uniform, non-linear etc) as the flow is not able to develop to a desired profile in such a short distance. This can negatively impact performance of the sensors or the accuracy of their measurements. Therefore it may be desirable to reduce this non-uniformity or turbulence to mitigate erroneous readings. To aid in reduction of turbulence upstream of the flow sensors, one or more flow conditioners may be provided as disclosed herein. One or more flow conditioners may be provided to condition the flow by making the flow more uniform over the cross-sectional area of the respective conduits leading up to the flow sensor. They may also aid in straightening the flow. A more uniform flow ensures the flow sensor is more likely to make readings that are representative of the behaviour of bulk flow travelling through the respective conduit

FIG. 6B shows an outlet connector 800 coupled with outlet 744 in the second component 720, and an O₂ connector coupled with O₂ inlet 712 in the first component 710. Also shown in FIG. 7A is a printed circuit board (PCB) 760 which may be screwed or press-fit or otherwise attached to the pneumatic block 700 (specifically, second block component 720) and contain processors and circuitry configured to control operation of one or more pressure sensors 114, 214, 414, as well as accommodating the pressure sensors themselves. PCB 760 may also contain processors and circuitry configured control operation of other electronic components of the device, including the blower 310, sensors 218, 318, 418 and proportional valve 212 although in some embodiments this functionality may be provided by processors and circuitry provided on a separate, larger PCB which may in some instances house the ambient pressure sensor 114. In some cases, one or more pressure sensors (such as O₂ pressure sensor/s 214, mixed gas pressure sensor/s 414) or one or more temperature sensors may be mounted to the PCB 760 and are provided through holes in the second component 720 to sense pressure in the relevant flow paths. It is to be understood that other similar sensors may be accommodated on the PCB if there is enough available space on the PCB. To reduce the risk of gas leakage, one or more gaskets, seals or O-rings 755 may be provided between the pressure sensors and/or flow sensors and the second component 720. In some embodiments, one or more O₂ pressure sensors 214 and/or one or more flow sensors may be provided to determine if an oxygen supply 210 has been connected to the oxygen inlet 712. In some embodiments, there may be two pressure sensors in the mixed gas flow path 400, one of which may be redundant and provided as a back up sensor in the event that the first mixed gas pressure sensor 414 fails. In some embodiments where there are two or more pressure sensors, one pressure sensor may operate over a different pressure range from another pressure sensor, for example one sensor providing accurate sensing at high pressures and another sensor providing accurate sensing at low pressures.

FIG. 7A is a transparent front view of the pneumatic block 700 of FIGS. 6A and 6B showing second block component 720 and third block component 730 and various features of the device arranged between those components. Second block component 720 contains three through bores 722, 723, 724 which may be arranged vertically and adjacent to one another, and each define part of the O₂ flow path 200, the air flow path 300 and the mixed gas flow path 400. Arrangement of the through bores 722, 723, 724 enable the pneumatic block 700 to be provided in a compact form factor.

Third component 730 also contains three through bores 732, 733, 734. Ideally these are also vertical and adjacent to one another and arranged to align with the three corresponding through bores 722, 723, 724 in the second component 720 to define part of the O₂ flow path 200, the air flow path 300 and the mixed gas flow path 400. The third component 730 also provides a cavity 736 configured to define a space receiving part of the blower 310 (ideally the motor side of the blower), and also defining the mixing chamber 500. Since the space receiving the blower 310 and defining the mixing chamber 500 is formed by the cooperation of a cavity 736 formed in the third component 730 and a cavity 715 formed in the first component 710, a seal or gasket 752 may be provided to reduce the risk of gas leakage. In some embodiments, the cavity 715 in the first component 710 may be configured to house the impeller part of the blower 310, or a part thereof, and the cavity 736 in the third component 730 may be configured to house the motor part of the blower or a part thereof. Since the third component may be configured to house the motor component of the blower 310, it may be desirable for the third component 730 to provide a structure that is capable of ensuring stability of the device when the blower 310 is operated at high speed, i.e. high revolutions per minute (RPM). Thus, it may be desirable that the material of the third component is capable of withstanding cyclic loads applied by the blower 310 and not prone to fail due to fatigue. Examples of suitable materials which may comprise or form part of the third component 730 may include, but are not limited to brass, and/or stainless steel and/or Aluminium alloy and/or anodized Al alloy and stainless steel. In some embodiments, third component 730 may comprise a mixed gas flow conditioner 750 as will be explained below.

Also shown in FIG. 7A is the blower outlet 320, showing a location for exit of air from blower 310 into mixing chamber 500 according to certain embodiments of the disclosure. It is to be understood, as described previously, that the location of the blower outlet 320 relative to the O₂ outlet 220 and the mixed gas inlet 510 can be important in improving performance of the device both in terms of reducing or eliminating undesirable flows in the mixed gas flow path 400 and in the O₂ flow path 200.

FIG. 7B is a transparent perspective front view of the pneumatic block 700 of FIGS. 6A, 6B and 7A. This view is provided to show that through bores 722, 732 together define an O₂ flow path 200 to the O₂ outlet 220 which provides O₂ to mixing chamber 500; through bores 723, 733 together define an air flow path 300 to the air outlet 320 which provides air to the blower 310, and bores 724, 734 together define a mixed gas flow path 300 between the mixed gas inlet 510 of mixing chamber 500 to the mixed gas outlet 744 (see FIG. 6A). Also shown in FIG. 7B is an outlet connector 800 on second component 720 and an O₂ coupling 980 provided on the rear of block 700 in first component 710 to couple O₂ inlet 712 with the O₂ supply 210. O₂ inlet 712 may be arranged to receive the O₂ coupling 980 in a direction that is orthogonal to the direction of the through bore 722 defining part of the O₂ flow path 200.

In some embodiments the O₂ inlet 712 is configured to provide O₂ conduit coupling such as a Diameter Index Safety System (DISS) type connection of standard CGA V-5:2019 although other connection types may be used depending on system requirements. The O₂ inlet 712 is provided at the rear of the block 700 and receives O₂ from O₂ supply 210. In some embodiments, the O₂ inlet 712 may be arranged to receive the coupling for an O₂ supply conduit when inserted with a force applied perpendicular to the rear face of the first component 710 of block 700. This arrangement may allow for the user to easily insert the O₂ supply coupling into the O₂ inlet 712. An O₂ pressure sensor 214 may be provided to sense pressure of gas from the O₂ source 210. In some embodiments, the O₂ pressure sensor 214 may be mounted on PCB 760 and sense pressure of the oxygen entering the inlet (upstream of the proportional valve). Seals, gaskets or O-rings 755 may be provided to reduce risk of gas leakage where the pressure sensor 214 is provided in the O₂ flow path 200. Flow sensors or other similar sensors may be positioned in the same, or substantially similar positions as the pressure sensor/s 214 described above.

FIG. 7B also shows proportional valve 212 arranged at the front of second component 720. O₂ received into O₂ inlet 712 passes through proportional valve 212 which expels the gas in the direction of flow sensor 218. The proportional valve is used to control the flow rate of O₂ in the O₂ flow path 200 and depending on the degree of openness of the valve, can cause high velocity and/or non-uniform flows to enter the flow sensor 218. In the sense that the proportional valve 212 modulates the flow of O₂ in the O₂ flow path 200, it may be considered a flow modulator. Thus, in some embodiments a flow conditioner may be provided to improve the uniformity of gas entering O₂ flow rate sensor 218 from the flow modulator provided by proportional valve 212.

In some embodiments, the O₂ flow conditioner may be described as a hybrid or two-stage flow conditioner 250 which is configured to condition the flow of O₂ gas as shown in FIGS. 18A to 18C. In some embodiments, the O₂ flow conditioner 250 comprises a first portion 250A configured to receive and disperse the flow of gases, and a second portion 250B configured to improve a characteristic (e.g. directionality such as straightness, spread across a cross-section of a flow path, uniformity in velocities, etc) of the dispersed gases received from the first portion. The first portion 250A comprises a porous component. The porous component may be a rigid component. The first portion 250A may be a filter. In some embodiments, the first portion 250A comprises a sintered metal filter, preferably a bronze sintered filter. The second portion 250B may comprise a flow conditioner having of a plurality of openings 252. The first portion 250A and the second portion 250B may be configured to cooperate, for example the first portion may comprise a conical outer shape made of porous material with a tip 255 that is shaped to key or cooperate with a correspondingly shaped recess 253 (which may be a blind bore or through hole) in the second portion 250B. A coupling of this type saves space within the device however it is to be understood that in other embodiments, the second portion 250B may be formed integrally with the second component 720 of the pneumatic block by machining, milling, drilling or the like. In some embodiments, the first and second portions 250A, 250B may be spaced apart and may not key or cooperate together. In some embodiments, a hybrid or two-stage flow conditioner 250 as disclosed herein may be beneficial by facilitating conditioning of flow toward a desirable flow profile in a shorter flow path than single stage flow conditioners that provide a single conditioning function, e.g. straightening of a flow. In some embodiments involving a focussed stream of gas, a longer entry flow path may be required so that the gas flow can develop before straightening in a flow conditioner corresponding to second portion 250B, to avoid the focussed gas flow flowing largely through the central portion of second portion 250B. A two-stage flow conditioner 250 as disclosed herein, disperses the focused flow before it is enters the second portion 250B and may have the benefit of shortening the gas flow path upstream of the second portion 250B providing flow straightening. This in turn has benefits in assisting to design flow systems having a smaller form factor. The hybrid or two-stage flow conditioner 250 may be arranged to receive a high velocity gas, e.g. downstream of proportional valve 212. In some embodiments, the conditioner 250 may be arranged downstream of the blower outlet 320.

In some embodiments, the first portion comprises a sintered filter having outer and inner profiles comprised of e.g. sintered bronze. As O₂ fills the inner cavity of the filter, the pressure inside increases. When the pressure exceeds a given threshold, the O₂ exits the filter through the porous sintered walls and disperses the O₂ all around the filter. The second element of the flow conditioner improves a characteristic of the dispersed flow, e.g. straightening the flow and/or increases uniformity across the cross-sectional area of the inlet to the O₂ flow sensor 218.

The plurality of openings in the second portion 230 of O₂ flow conditioner may comprise a circular, oval, elliptical, hexagonal or other cross sectional shape, or a combination of these as illustrated in FIGS. 5A to 5C. In some embodiments the second portion 230 of the O₂ flow conditioner comprises a plurality of parallel flow channels 232. In some embodiments, a honeycomb cross-sectional profile in which the plurality of flow channels are hexagonal in cross section. The flow channels may be of equal length, or they may each be of different lengths. In some embodiments, the plurality of flow channels are of non-uniform diameter as shown, and may be arranged radially in the second portion. Ideally, the plurality of flow channels are arranged in the second portion such that they are entirely within the bounds of a flow channel downstream of the flow conditioner, such as a flow channel feeding into the O₂ flow sensor 218. In some embodiments, the second portion 230 may be a separate part that is inserted into the O₂ flow path 200. A groove 234 may be provided in an external wall of the second portion 230 to receive a seal, gasket or O-ring configured to minimise O₂ leakage between the second portion and the through bore 722 defining the O₂ flow path.

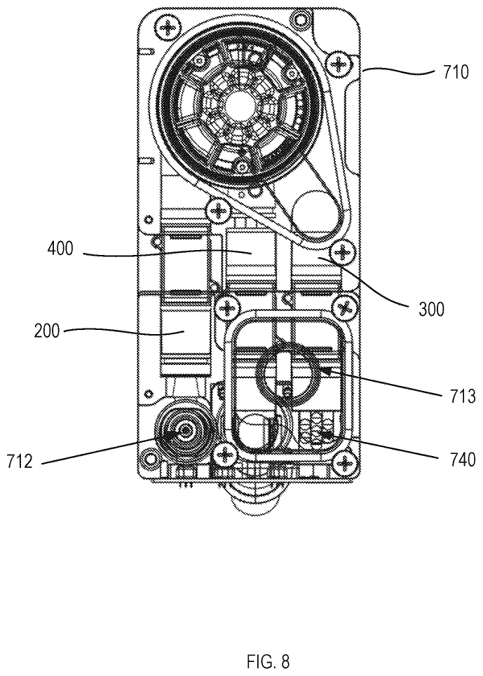

FIG. 8 is a transparent rear view of the pneumatic block 700 in FIGS. 6A to 7B showing in particular features of first component 710. An air inlet 713 receives ambient air into the rear of the first component 710. The ambient air may first pass through a filter which may be placed over air inlet 713 and/or a housing in which pneumatic block 700 is contained. Air inlet 713 receives air into a cavity 714 formed in the first component 710 which may have an overall square, rectangular, obround, oval, circular or other cross sectional shape. Air inlet 713 is arranged to receive air in a direction that is orthogonal (i.e. 90°) to the direction of the through bore 733 defining part of the air flow path 300 and containing air flow rate sensor 318. As the air flow turns from inlet 713 into bore 733, the change in flow direction can cause the flow to separate from the walls defining the flow path.

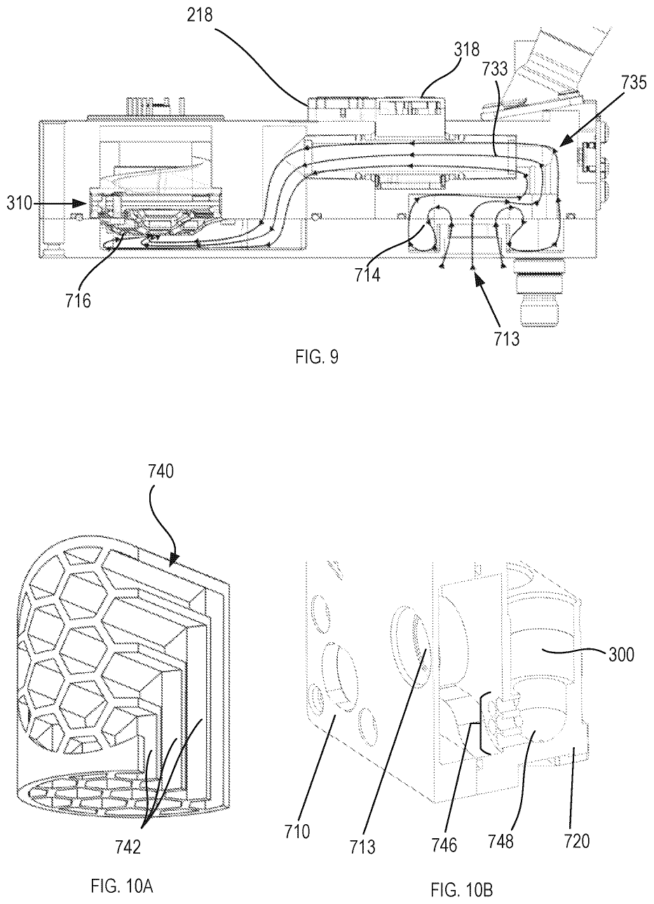

FIG. 9 is a flow diagram showing streamlines of air received in the air inlet 713 and air cavity 714 of first component 710 and traveling through bore 723 in the second component 720 and through bore 733 in the third component 730 into a cavity 716 in the first component 710 that supplies air to the inlet of blower 310. Much of the turning flow utilizes a larger turning radius at the outer edge of the 90° corner 735. This can cause flow that is faster moving to be at the top of the flow path when entering the air flow rate sensor 318. Toward the bottom of the flow path, particularly toward the inside of the 90° corner 735 there is little to zero flow which may be attributable to the air having too much energy to turn in the small radius. Thus, while the corner 735 enables the air flow path to be built into the pneumatic block 700 with a compact form factor, it can create non-uniform flow in the vicinity of the flow sensor 318 which can result in inaccurate readings. In some embodiments, this may be mitigated by providing a flow conditioner 740 in the air flow path 300.

FIG. 10A is a schematic illustration of an air flow conditioner 740 that may be provided in air flow path 300. As in the embodiment shown, air flow conditioner 740 may comprise a 90° flow conditioner that is configured to be provided in the 90° turn in the flow path 300 at corner 735. Ideally, the internal structure of air flow conditioner 740 comprises a plurality of flow channels 742 configured to receive air from air inlet 713 and provide parallel flow paths through the corner 735 such that air flow exiting the flow conditioner 740 into the through bore 733 is substantially uniform. Thus, the air flow conditioner 740 may receive and maintain uniform flows in air entering air inlet 713, or it may improve uniformity of air exiting the air inlet 713 that may not have had good uniformity upon entry. The flow channels 742 within air flow conditioner 740 may comprise a circular, oval, elliptical, hexagonal (as shown) or other cross sectional shape, or a combination of these. For instance, a honeycomb cross-sectional profile in which flow channels 742 are hexagonal may provide beneficial pressure profiles across the air flow conditioner 740 since they provide a larger cross-sectional area for the air to travel across. In some embodiments, the flow channels 742 are arranged within air flow conditioner 740 so that flow exiting the flow channels 742 is unobstructed as it enters through bore 733. Provision of air flow conditioner 742 may improve uniformity of flows into air flow sensor 318 which may improve overall performance of the system. It is to be understood that air flow conditioner 740 may be provided as a separate component of the pneumatic block 700 or it may be formed integrally with the second component 720 of block 700 e.g. by machining, milling, additive manufacturing, 3D printing or the like.

FIG. 10B is a schematic illustration of another air flow conditioner 746 that may be provided in air flow path 300. As in the embodiment shown, air flow conditioner 746 is integral with the second block component 720. Air flow path 300 includes a corner 748 which may be radiused to minimize pressure drop as flows travel around the corner 748. The corner 748 may be positioned downstream of the flow conditioner 746. The air flow conditioner 746 conditions flows (e.g. spreads out the flow across the cross-sectional area of the flow path) travelling from cavity 714 to air flow rate sensor 318. The provision of air flow conditioner 746 may improve the uniformity of flows travelling through air flow sensor 318 which may improve accuracy of the flow sensing by air flow rate sensor 318. It is to be understood that air flow conditioner 746 may also be provided as a separate component of the pneumatic block 700 (not shown) or it may be formed integrally with the second component 720 of block 700 (as shown) e.g. by machining, milling, additive manufacturing, 3D printing or the like, as shown in FIG. 10B.

In some embodiments, blower 310 comprises a centrifugal blower configured to draw air in from air inlet 713 through a central inlet on one side of the blower positioned in cavity 716 in first component 710. The blower 310 may propel the gas tangentially into a spiral shaped cavity of the blower towards the blower outlet 320 as discussed above. In some embodiments, air exits the blower outlet 320 tangentially. The blower 310 moves air within the pneumatic block 710 predominantly by suctioning it from the air inlet 713. O₂ may be introduced to the gas flow generated by the blower 310 downstream of the blower. Owing to the downstream introduction of O₂, the blower does not impart energy to O₂ in the mixed gas flow since the O₂ gas is not moved by the blower's blades. Consequently, the blower 310 moves less volume of gas than systems that mix air with O₂ upstream of the blower, which may require less power and generate less heat. Additionally, the compressed O₂ which is at a lower temperature than ambient air will absorb heat from the motor of blower 310. Alternatively/additionally, due to the conductive nature of the pneumatic block components in certain embodiments heat may dissipate by thermal transfer through one or more block components. Both of these may provide a cooling effect on the device. This may improve efficiency of operation and/or reduce the risk of the blower motor overheating. Additionally, mixing O₂ and air downstream of the blower 310 may provide a mixed gas flow that is of a lower temperature than systems that provide mixing of these gases upstream of the blower. A mixed gas flow of lower temperature may be beneficial in embodiments where the mixed gas flow is humidified and/or warmed before it is provided to the patient since this enables more accurate control over the heating and humidification of gases by a downstream humidifier. In some embodiments, the turbulence of the air stream exiting the blower 310 at blower outlet 320 and/or the turbulence of O₂ exiting the O₂ flow path 200 at O₂ outlet 220 cause mixing of gases within the mixing chamber 500 defined by the cooperating cavities 715 and 736.

When the blower 310 is mounted between first component 710 and third component 730 of a pneumatic block 700, it may be desirable to provide at least three seals. A first seal or gasket 752 may be provided between the first component 710 and the third component 730 to reduce risk of gas leakage. This seal may also isolate vibration generated by the blower 310 during operation, minimising the likelihood of vibrations transferring through the pneumatic block 700 to the mounting element 701 and equipment to which the device is mounted. This seal may be configured to limit leakage of air and/or O₂ from one or both of the cooperating cavities 715 and 736. Leakage of O₂ and air from the mixing chamber (and/or joins within the pneumatic block 700) may provide a fire risk and may also present a risk of pressure loss which is undesirable. This seal may also function to limit incursion of air or other gases that may exist inside the housing from entering the mixing chamber. Moreover, this seal may beneficially prevent the excursion of gases from the mixing chamber to the surroundings. A second seal 753 may be provided at the outer circumference of the blower 310. The second seal 753 may mount the blower 310 to one or both of the first component 710 and third component 730. The second seal 753 may be clamped between a portion of the first component 710 and a portion of the third component 730 to secure the blower 310 within the cavity formed by the first and third components 710, 730. Seal 753 may also isolate vibrations arising from operation of the blower 310. A third seal 756 may be provided around the blower motor assembly. The third seal 756 may also act to isolate and reduce transfer of vibrations from the blower motor assembly to the third component 730. The third seal 756 may prevent the excursion or incursion of gases to or from the mixing chamber 500.

In some embodiments, the first component 710 is configured to perform a significant load bearing function of the device. In some embodiments, first component 710 may comprise a mounting element 701 that may be configured to cooperate with a mounting structure such as a mount bracket, pole mount or monitor mount. Alternatively or additionally the mounting element may be attached to the rear casing of the device. The mounting element 701 may be formed of one or more parts. In some embodiments, the mounting element 701 or a part thereof may be directly coupled with the first component 710. In some embodiments the mounting element 701 or a part thereof (best shown in FIGS. 17A and 17B) may be interchangeable and switched out by a user according to specific needs. In some embodiments, a mounting element 701 comprising a monitor mount may enable the device to be mounted to e.g. an anaesthesia or other machine configured to provide certain types of respiratory support or other therapy to a patient. Ideally the various mounting methods that may be effected by the mounting element 701 enable the device to be customized to environment in which the patient is being treated, ideally to ensure visibility while also providing a stable, secure and effective platform for delivering respiratory support. In some embodiments, the mounting element 701 may be provided through a housing in which the pneumatic block is provided.

In some embodiments, blower 310 expels flow such that gases within the mixing chamber 500 (which may comprise one or both of O₂ and air) are moved by the flow generated by the blower 310 and/or by flow through the O₂ flow path. In some embodiments, the flow which exits the blower 310 (i.e. expelled) comprise high velocity flows and/or are turbulent. In embodiments where O₂ from the O₂ flow path and air from the blower 310 is entering the mixing chamber 500, the O₂ and air travel in the mixing chamber 500 in a way that they combine to form mixed gases. While the high velocity and/or turbulent flows generated by blower 310 may be effective in mixing gases, these flow characteristics can cause inaccurate sensing of mixed gas flow rates by mixed gas flow sensor 418 provided in the mixed gas flow path 400. This is partly because the mixed gas flow may not comprise a desirable flow profile across a cross-section of the mixed gas flow 400 that allows for accurate sensing by the mixed gas flow sensor 418. A desirable flow profile depends on the application. A desirable flow profile may be parabolic. A parabolic flow profile is typical of laminar flows through circular pipes while a profile with a flat or flatter leading edge is typical of turbulent flows. To mitigate undesirable flows, in some embodiments, the present disclosure may provide a mixed gas flow conditioner 750 to improve uniformity of mixed gases before they enter the mixed gas flow sensor 418. The mixed gas flow conditioner 750 may be provided at or near the mixed gas inlet 510 and may straighten the flows, spread the flows across the mixed flow path 400 and/or break up large eddies that can develop in the mixed gas flow. In some embodiments, this is achieved by providing a mixed gas flow conditioner 750 having a plurality of flow channels 751. As the flow of mixed gases from the mixing chamber 500 exits through mixed gas inlet 510, it is forced through the smaller channels 751, increasing uniformity of the flow.

The flow channels 751 within mixed gas flow conditioner 750 may comprise a circular, oval, elliptical, hexagonal or other cross sectional shape, or a combination of these. The cross sectional shape need not be the same, or of the same dimension, for all flow channels 751. Although circular flow channels 751 are shown in FIG. 11A, a honeycomb cross-sectional profile in which flow channels 751 are hexagonal may provide a lower pressure drop across the air flow conditioner 750 since they provide a larger cross-sectional area for the air to travel across. In some embodiments, the flow channels 751 are arranged within air flow conditioner 750 so that flow exiting the flow channels 751 is unobstructed as it enters mixed gas flow sensor 418. Thus, the flow channels 751 may be arranged within the bounds of the flow conditioner 750 such that they are arranged also within the internal bounds of the inlet to the mixed gas flow sensor 418. Providing for unobstructed flow from the flow channels 751 to the mixed gas flow sensor 418 may avoid recirculation of gas and/or loss of uniformity which can have negative consequences on accuracy of flow rate measurements. Provision of the mixed gas flow conditioner 750 may improve uniformity of flows into mixed gas flow sensor 418 which may improve overall performance of the system. While flow conditioner 750 is described in the context of improving uniformity of flows in the mixed gas flow path, it is to be understood that such a flow conditioner may also be used in the air and oxygen gas flow paths.

Mixed gas flow conditioner 750 may be provided as a separate component of the pneumatic block 700 as illustrated in FIG. 7B and FIGS. 11A and 11B, or it may be formed integrally with the third component 730 of block 700 e.g. by machining, milling, drilling or the like as illustrated in FIG. 7A. Where mixed gas flow conditioner 750 is a separate component, a groove 754 may be provided to receive a seal, gasket or O-ring configured to minimise mixed gas leakage between the mixed gas inlet 510 and the mixed gas flow sensor 418.

In embodiments where the mixed gas flow conditioner 750 is formed integrally with the third component 730 of block 700 (see FIG. 7A), the flow channels 751 may be drilled or otherwise formed to form the length of the flow conditioner which terminates at the mixed gas inlet 510 of the mixing chamber 500. In some arrangements, the mixed gas inlet 510 is arranged to be continuous with a wall of the mixing chamber, for example aligned with an arc of a side wall of the mixing chamber. This enables the inflow end of the channels 751 to conform with the arc-shaped internal contour of the mixing chamber. This may reduce or eliminate undesired flow that may occur when gases in the mixing chamber collide with an edge of the flow conditioner, which may be likely if the flow conditioner 750 is formed as a separate component that comprises a portion that protrudes into the mixing chamber when assembled with the block 730. Hence an integral formation of the flow conditioner 750 with block 730 is advantageous.

In preferred embodiments, the mixed gas flow conditioner 750 has a length sufficient to generate flows with enough uniformity that erroneous flow rate readings by mixed gas flow sensor 418 are reduced or eliminated. In some embodiments, the mixed gas flow conditioner 750 may have a length that spans a distance between the mixed gas inlet 510 and an inlet to mixed gas flow sensor 418, as illustrated in 7A.

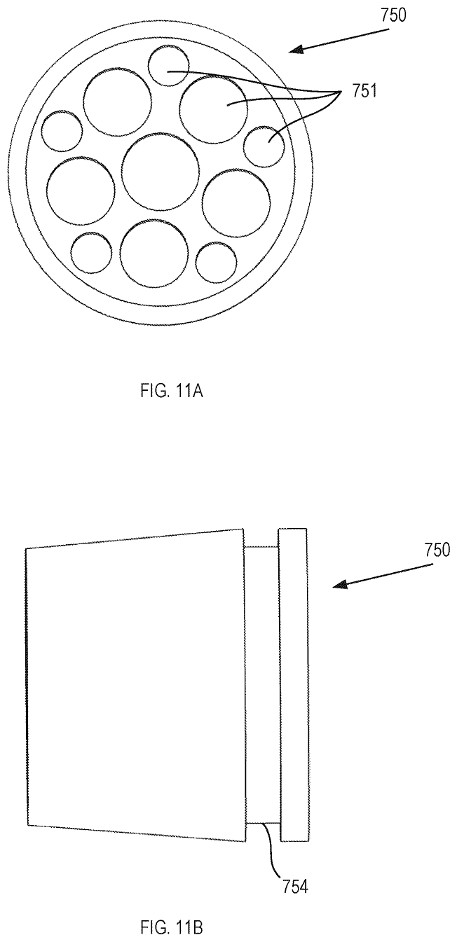

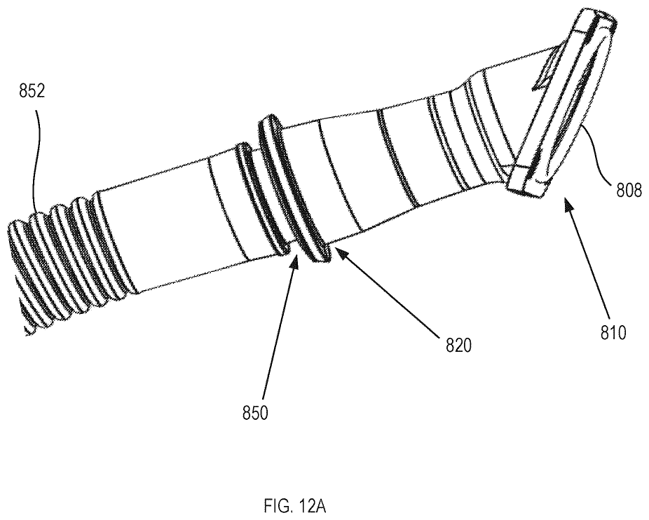

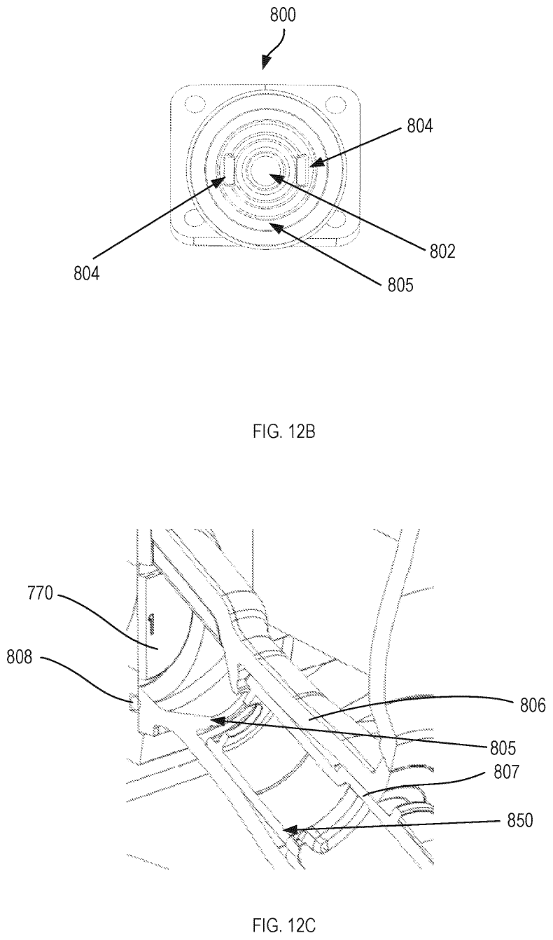

FIG. 12A is a side view of an outlet connector 800 according to embodiments of the disclosure. Outlet connector 800 may be oriented to receive the delivery connector at an angle requiring application of a connection force simultaneously in the vertical (upward) and horizontal (lateral) directions. For example, the outlet connector may be oriented at an angle of approximately 60 degrees to vertical. FIG. 12B is an end view of connector 800 from an inlet end 810. FIG. 12C is a sectional view of outlet connector 800 showing internal features according to certain embodiments. In FIG. 12A connector 800 is shown coupled (for example by an interference fit) with a delivery connector 850 which provides a fluid flow path via delivery conduit 852 for gases to be provided to the patient by a patient interface. In some embodiments the fluid flow path may also comprise a humidifier. Outlet connector 800 has a larger central opening 802 which may be aligned with a corresponding central opening in delivery connector 850. The outlet connector 800 has a plurality of smaller apertures such as apertures 804 as shown in FIG. 12B. the apertures 804 may be provided toward a middle portion of the length of outlet connector 800 as shown. In some embodiments the plurality of apertures may be arranged closer to a central axis of the outlet connector than they are to a periphery of the outlet connector. These smaller apertures 804 increase the total cross sectional area of the openings between inlet end 810 and outlet end 820 of the outlet connector 800 through which gas from the mixed gas flow path 400 may flow to enter the delivery connector 850 and conduit 852. This maximizes the amount of flow from the outlet connector 800 to the delivery connector 850 and delivery conduit 852. In some embodiments, the smaller apertures 804 are configured to cooperate with corresponding openings in a specially designed delivery connector 850 which also has smaller apertures (not shown) arranged around a periphery of a main flow path within the delivery connector. An example of a delivery connector 850 may be a connector having features described in WO/2020157707, the entirety of which is incorporated by reference herein.

In some embodiments, the central opening 802 and the plurality of apertures 804 provide a plurality of flow paths when coupled with a delivery connector 850, comprising at least a central flow path through the main opening 802 and a plurality of outer flow paths through the smaller outer apertures 804. The outer flow paths may be substantially parallel to the central flow path which may improve efficiency of operation since the larger total opening area provided by the combination of the larger central opening 802 and the smaller apertures 804 (which may be matched by corresponding openings in the delivery connector 850) may result in a lower pressure drop across the connection. Additionally, the arrangement of the central opening 802 with the smaller apertures 804 reduces the risk of over-insertion of a delivery connector 850 into the outlet connector 800 since the tip of any such delivery connector would first collide with the flange or web portion 805 surrounding the central opening 802 and smaller apertures 804. The obstruction provide by web portion 805 may protect components upstream of the connector from over insertion of the delivery connector. Such upstream components may include a non-return valve 770 which may be provided to limit or prevent reverse flow of gases into the device as will be discussed below. Outlet connector 800 may have an internal taper 807 that guides the delivery connector 850 into position and provides a first sealing surface with the delivery connector 850. A further taper 806 provides a second sealing surface with the delivery connector 850 and may also provide a degree of protection against over-insertion of the delivery connector 850.

In some embodiments, a seal, gasket or O-ring 808 may be provided to form a substantially sealing join when the outlet connector 800 is fastened to a pneumatic block 700. The seal 808 may mitigate leaks and unnecessary pressure losses in the flow of gas provided to the patient which may further improve operating efficiency.

A portion of the outlet connector 800 between the plurality of apertures 804 and the outlet end 820 may provide a smaller internal cross-section at or near the plurality of apertures 804, relative to the internal diameter of the outlet connector at the terminal outlet end 820. That is, the outlet end 820 may have a smaller internal cross-section at or near the plurality of apertures 804 at or near a middle portion of the outlet connector 800 compared to an internal cross-section at or near the terminal outlet end.

In some embodiments, a non-return valve 770 prevents gases flow from the outlet connector 800 back into the device, in particular into the mixed gas flow path 400. The non-return valve 770 may comprise a weighted flap portion. The non-return valve 770 may be positioned at an angle, such as e.g. a 10° angle from vertical, such that gravitational forces act on the weighted flap portion and bias the non-return valve to a closed position when the device is oriented upright (normal operating position) and when there is no or substantially low flow. Orientation of the outlet connector A flow travelling from the device towards the outlet connector 800 with a sufficient force that overcomes the weight of the flap portion would actuate open the non-return valve 770. A reverse flow travelling in a direction from the outlet connector 800 towards the device, in particular the mixed gas flow path 400 would bias close the non-return valve 770.

In some embodiments, the device may comprise the pneumatic block 700 as described elsewhere herein and the outlet connector 800 may be fastened or coupled to the pneumatic block. In some arrangements, mixed gas from the mixed gas flow path 400 turns a corner such as a 90° corner before it travels to the outlet connector 800. The outlet connector 800 may be fastened to the pneumatic block 700 so that it is oriented at an angle such as e.g. a 60° angle to vertical, to receive the delivery connector 850 at an angle that requires application of both lateral and upward connection forces in order for the delivery connector to form a substantially sealing coupling when received inside the outlet connector 800. The non-return valve 770 comprises a mounting portion that is retained between the rib or protrusion of the outlet connector 800 and a portion of the block 700. As explained above, the non-return valve 770 may be opened by the flow of gases from the mixed gas flow path 400 travelling towards the outlet connector 800 however flow in the opposite direction (i.e. from outlet connector 800) actuates the non-return valve closed. This may prevent ingress of contaminants or water or water vapour (from a downstream humidifier) which can cause deterioration of the pneumatic block, compromise a sterile environment, and/or damage components of the device.

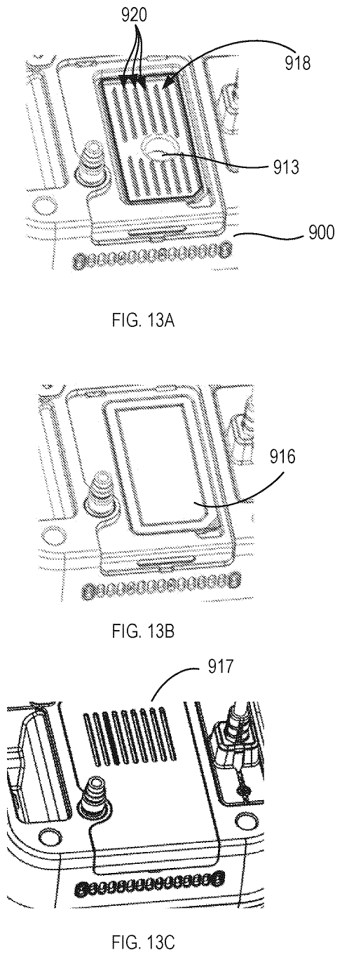

The device may be provided with a housing forming an outer casing. The housing may be moulded from a polymer (for example polycarbonate) and/or formed from another material. The housing material may provide flame retardancy, such that in the event of a fire, the housing may self-extinguish the fire. Ambient air is drawn into the housing through an air inlet 913 and continues along the air flow path 300 towards the blower 310. The air inlet may comprise a removable filter 916 with a removable filter cover 917 as shown in FIGS. 13A to 13C. The filter 916 may cover a recess 918 in the housing 900 which comprises a plurality of ribs 920 which act as spacers to maintain the filter 916 at a distance from the surface of the housing in the recess. The troughs between ribs 920 and the filter 196 create channels that enable air to freely flow into the air inlet 913.

In some cases, the device contained within housing 900 may be operable to supply 100% O₂ to a patient via outlet connector 800. However, a leak from any flow path within the device may cause O₂ to flow or accumulate inside the housing which may provide a risk of fire. Therefore, in some embodiments it is desirable to prevent accumulation of O₂ within the housing. This may be achieved, in some embodiments, by use of a ventilation fan or blower 650 which is distinct from the blower 310.

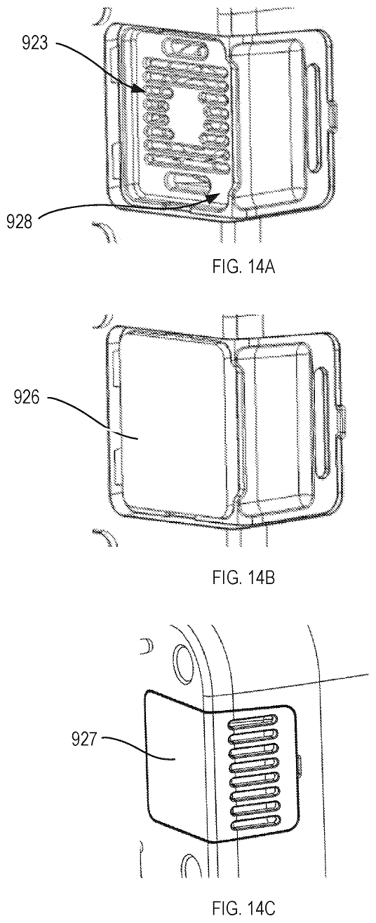

Ambient air may be drawn into the housing to the ventilation blower 650 through a second inlet 923. The second air inlet 923 may comprise a separate removable filter 926 with a removable filter cover 927 as shown in FIGS. 14A to 14C. The filter 926 may cover a recess 928 in the housing 900 which receives the filter 926. The filter cover 927 may be a snap-fit or press-fit, screwed or otherwise fastened in position in a manner which enables removal (e.g. by levering off with a flat screw head or other tool) to give access to the filter 926 for cleaning, replacement and the like. Ideally, air drawn in through second inlet 923 is drawn into the housing 900 by the ventilation blower 650 and may be distributed within the housing to minimise dead space. This may reduce or prevent O₂ accumulation in the bousing.

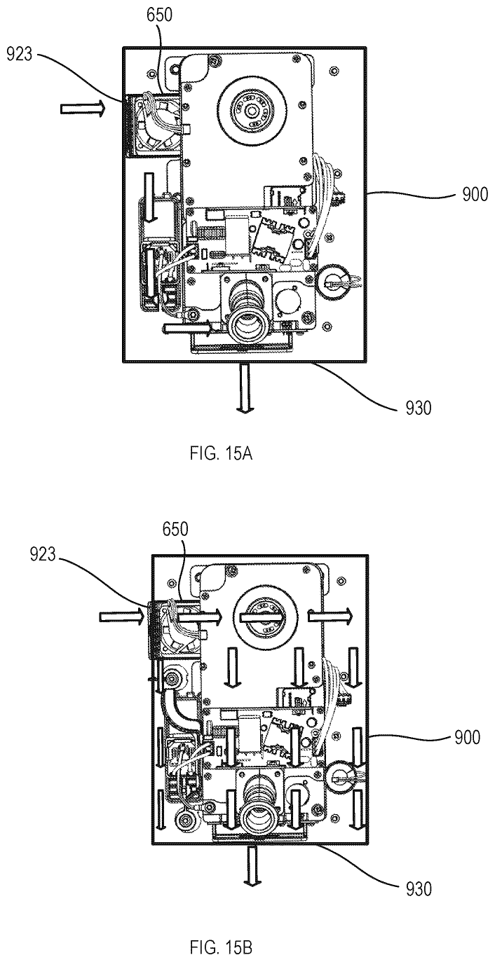

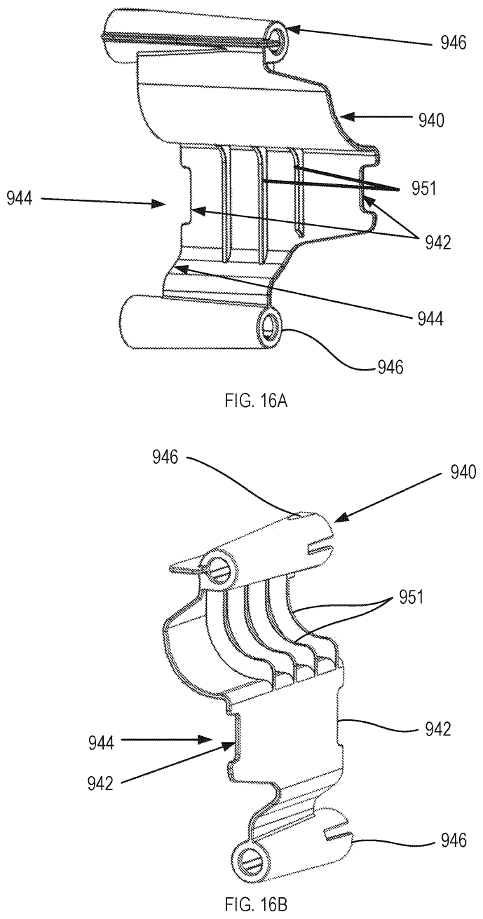

Typically, flow within the housing will take a path of least resistance as exemplified by the arrows in FIG. 15A. However, it is preferred that the flow inside the housing is directed to minimise O₂ accumulation. FIG. 15B shows a more preferable flow path for air entering inlet 923, wherein the flow is directed substantially across the width of the device before it travels downward to the air outlet 930. Flows closer to the ventilation blower 650 may travel with a faster velocity, with the air flow slowing as it travels toward air outlet 930.

In some embodiments, one or more baffles 940 may be provided to direct flow within the housing, examples of which are provided in FIGS. 16A and 16B and FIGS. 17A and 17B. Baffles may be manufactured from an elastomeric material comprising silicone and/or other materials that may be e.g. compression moulded, or from other suitable materials such as e.g. injection moulded thermoplastic elastomer which may impart rigidity where required. The baffle 940 may be positioned in compression between front and rear covers comprising the housing 900. Owing to the flexibility of the baffle material, the structure of baffle 940 may compress and conform to the internal contours of the front and rear covers. Advantageously, the compliant baffle material may also act as an attenuator for vibrations and/or noise generated by the ventilating blower 650 and/or the flow generating blower 310.

The baffle 940 may be configured with one or more cut out portions 942 to provide clear passage for a connector and wiring that is required inside the housing 900 to power the ventilating blower 650 and the flow generating blower 310 as well as PCB 760 from a power distribution board of the device (not shown). A contour or cut out portion 944 allows air from the ventilating blower 650 to enter part of the housing 900 that contains the IEC connector. Ribs 951 provide structural integrity and may prevent baffle 940 from sagging (due to gravity and/or material degradation). Additionally, ribs 951 may aid in splitting and/or guiding air flow within the housing e.g. over the power distribution board.

While ventilating inside the housing 900 may reduce accumulation of O₂, it may also have a beneficial effect providing a mechanism for thermal regulation by removing heat from inside the housing. This may further improve efficiency of operation and reduce the risk of components in the device overheating.

The baffle 940 may comprise one or more hollow conical portions 946 that may be configured to each fit over a screw boss in the housing to locate the baffle in position. Various features may be provided in the baffle 940 to cooperate with components in the housing 900 to limit movement including one or more of slipping and twisting when the baffle becomes compressed between front and rear covers of the housing. These may include e.g. slots at the base of the baffle 940 that mate with a rib on the rear housing. In some embodiments, it may be desirable to avoid use of screws or other fasteners being applied though the baffle 940 as these can create areas of localised stress which may lead to material failure e.g. by cracking or splitting upon assembly or over time. FIGS. 17A and 17B show an example of baffle 940 arranged to direct flows of air from ventilating blower 650 across pneumatic block 700 and over IEC connector. FIG. 17B shows the arrangement of FIG. 17A, further showing a display screen 20 providing part of an I/O device used to receive user inputs for determining operational parameters of the device, and power distribution board 970.

Embodiments of the present disclosure provide a device for delivering respiratory gases at an O₂ concentration that is higher than ambient air in a manner which may be safer and more efficient than existing devices. Owing to the arrangement of flow paths and their inlets and outlets within the device relative to a blower, undesirable flows may be reduced or avoided which can improve sensor accuracy, while also achieving gas mixing when a mix of O₂ and air is required to be delivered. In some embodiments, the device is provided by a pneumatic block comprised of a plurality of block components which contain cooperating bores and cavities that define flow paths. In some embodiments the block components are arranged with one or more flow sensors and one or more pressure sensors that may be configured to safely and reliably deliver the required flow of respiratory gases. Meanwhile, the arrangement of the bores and cavities in the block components can provide a compact device with a form factor that may be beneficial particularly in medical environments where space for additional apparatus may be limited.

Additionally various features such as flow conditioners, filters, outlet connectors and baffles may be provided to enhance overall operation of the device providing potential for improvements in operational efficiency and/or accuracy and/or safety.

The device generating a flow of respiratory gases as disclosed herein may have utility in delivering high flows of respiratory gases to a patient. In particular, the device may be of utility during anaesthetic procedures although use of the device is not limited to such procedures and may be used to deliver respiratory gases comprising air, 100% O₂ or a mix of air and O₂ in other environments such as in ICU or other medical environments where a patient requires high flow respiratory support. Operational parameters of the device may be controlled to suit the respiratory support requirements (such as e.g. gases composition, flow rate and/or pressure) of the patient by use of one or more sensors, controller 4, blower 310 and other features of the device as disclosed herein.

It is to be understood that various modifications, additions and/or alternatives may be made to the parts previously described without departing from the ambit of the present disclosure as defined in the provisional claims appended hereto.

The disclosure may also be said broadly to consist in the parts, elements and features referred to or indicated in the specification of the application, individually or collectively, in any or all combinations of two or more of said parts, elements or features. Where, in the foregoing description reference has been made to integers or components having known equivalents thereof, those integers are herein incorporated as if individually set forth. Similarly, where in the foregoing description reference has been made to features or elements of a particular aspect or embodiment, it is to be understood that those features or elements are herein incorporated as if expressly disclosed in combination with other aspects or embodiments for which a skilled addressee would appreciate those features or elements to be compatible.

Where any or all of the terms “comprise”, “comprises”, “comprised” or “comprising” are used in this specification (including the provisional claims) they are to be interpreted as specifying the presence of the stated features, integers, steps or components, but not precluding the presence of one or more other features, integers, steps or components or group thereof.

Future patent applications may be filed i on the basis of or claiming priority from the present application. It is to be understood that the following provisional claims are provided by way of example only, and are not intended to limit the scope of what may be claimed in any such future application. Features may be added to or omitted from the provisional claims at a later date so as to further define or re-define the invention or inventions.

1. A device for providing respiratory gases, the device comprising:
(a) a blower configured to receive a first gas from a first gas flow path and generate a flow of a first gas provided through a first gases outlet of the blower;
(b) a second gas flow path configured to receive a flow of a second gas and provide the flow of the second gas through a second gases outlet; and
(c) a mixing chamber configured to receive a flow of the first gas from the first gases outlet and a flow of the second gas from the second gases outlet, the received gases configured to mix in the mixing chamber to form a mixed gas, the received gases configured to travel in a mixing flow direction in the mixing chamber towards a mixed gases inlet, wherein mixed gas exits the mixing chamber via the mixed gases inlet providing flow to a mixed gas flow path.

2. The device according to claim 1, wherein the mixing chamber receives the flow of gases from the second gases outlet upstream of the first gases outlet in the mixing flow direction.

3. The device according to claim 1 or claim 2, wherein one or both of the first gases outlet and the second gases outlet are arranged to achieve flow of the first gas and/or the second gas substantially tangentially along a wall of the mixing chamber.

4. The device according to any one of the preceding claims, wherein the first gases outlet and the second gases outlet are arranged relative to the mixing chamber such that the first gases outlet is arranged to direct the first gas entering the mixing chamber away from the second gases outlet.

5. The device according to any one of the preceding claims, wherein the first gases outlet and the second gases outlet are arranged such that the first gas in the first gases outlet is directed in a first flow direction between a direction that is substantially parallel to a second flow direction of the second gas in the second gases outlet, and a direction that is substantially perpendicular to the second flow direction.

6. The device according to claim 5, wherein the first flow direction relative to the second flow direction is at an angle of about 0° to less than about 90°.

7. The device according to claim 5 or claim 6, wherein the first flow direction and the second flow direction are in a common plane.

8. The device according to any one of claims 5 to 7, wherein the mixed gases inlet is arranged such that the mixed gas flow in the mixed gas flow path is directed in a mixed flow direction between a direction substantially perpendicular to one or both of the first and second flow directions and a direction anti-parallel to one or both of the first and second flow directions.

9. The device according to claim 8, wherein the mixed flow direction, the first flow direction and the second flow direction are in a common plane.

10. The device according to claim 8 or claim 9, wherein the mixed flow direction is substantially anti-parallel to the second flow direction.

11. The device according to any one of the preceding claims, wherein the mixing chamber is substantially circular in cross section.

12. The device according to any one of the preceding claims, wherein the mixing chamber is substantially cylindrical.

13. The device according to any one of the preceding claims, wherein the mixing flow direction in the mixing chamber is around a central shaft.

14. The device according to claim 13, wherein the central shaft comprises a portion of the blower.

15. The device according to any one of the preceding claims, wherein the mixing chamber is configured such that gases travel in the mixing chamber in a spiral manner.

16. The device according to any one of the preceding claims, wherein the mixing chamber is comprised of a plurality of adjacent sectors, and wherein the first gases outlet and the second gases outlet are arranged in adjacent sectors of the mixing chamber.

17. The device according to claim 16, wherein the mixed gases inlet is arranged in a non-adjacent sector to the first gases outlet.

18. The device according to claim 16 or claim 17, wherein the mixed gases inlet is provided in a sector that achieves optimal uniformity of flow within the mixed gases flow path.

19. The device according to any one of claims 16 to 18, wherein the plurality of sectors comprises four quadrants.

20. The device according to any one of the preceding claims, wherein arrangement of the second gases outlet permits flow of gases from the mixing chamber into the second gas flow path.

21. The device according to any one of the preceding claims, wherein the second gases outlet comprises a lead-in portion.

22. The device according to claim 21, wherein the lead in portion comprises a taper configured to direct flow of the second gas into the mixing chamber.

23. The device according to any one of the preceding claims, wherein the second gas flow path comprises a flow conditioner at the second gases outlet which is configured to increase resistance to flow of gases from the mixing chamber.

24. The device according to claim 23, wherein the flow conditioner comprises a plurality of substantially parallel flow channels.

25. The device according to claim 23 or claim 24, wherein the flow conditioner has an outlet end that is shaped to be continuous with an internal wall of the mixing chamber.

26. The device according to any one of 23 to 25, wherein the flow conditioner is formed integrally with the device.

27. The device according to any one of the preceding claims, wherein the second gas flow path comprises one or more nozzles configured provide the flow of the second gas to the mixing chamber through a nozzle diameter which is less than a diameter of the second gas flow path.

28. The device according to any one of the preceding claims, wherein the second gas flow path comprises a non-return valve.

29. The device according to any one of the preceding claims, wherein the device comprises a first flow sensor for sensing flow rate of gases in the first gas flow path.

30. The device according to any one of the preceding claims, wherein the device comprises a second flow sensor for sensing flow rate of gases in the second gas flow path.

31. The device according to any one of the preceding claims, wherein the second gas flow path comprises a proportional valve.

32. The device according to claim 31, wherein the second flow sensor senses flow rate of gases downstream of the proportional valve.

33. The device according any one of the preceding claims, wherein the device comprises a third flow sensor for sensing flow rate of the mixed gas in the mixed gas flow path.

34. The device according to claim 33, wherein the mixed gas flow path comprises a mixed flow conditioner upstream of the third flow sensor.

35. The device according to claim 34, wherein the mixed flow conditioner is located at or proximal to the mixed gases inlet.

36. The device according to claim 34 or claim 35, wherein the mixed flow conditioner has an inlet end that is shaped to be continuous with an internal wall of the mixing chamber.

37. The device according to any one of claims 34 to 36, wherein the mixed flow conditioner comprises a plurality of substantially parallel flow channels.

38. The device according to any one of claims 34 to 37, wherein the mixed flow conditioner is integral with the mixed gases inlet.

39. The device according to any one of the preceding claims, wherein one or more of the first gas, second gas and mixed gas flows comprise a flow rate of 0 L/min or greater, optionally the mixed gas flow comprises a flow rate of about 20 L/min to about 90 L/min, optionally the mixed gas flow comprises a flow rate of about 40 L/min to about 70 L/min.

40. The device according to any one of the preceding claims, comprising a plurality of cooperating components through which bores have been formed which cooperate to define a plurality of gas flow paths, and into which cooperating cavities have been formed to define a cavity for receiving the blower and a mixing chamber.

41. The device according to any one of the preceding claims, comprising a pneumatic block having three or more cooperating components, wherein:
(a) a first component comprises a first opening defining a first gases inlet, a second opening defining a second gases inlet, and a first cavity for receiving a first part of the blower;
(b) a second component comprises three parallel through-bores defining part of each of the first gas flow path, the second gas flow path and the mixed gas flow path, and a third opening defining a device outlet; and
(c) the third component comprises three parallel through-bores defining part of each of the first gas flow path, the second gas flow path and the mixed gas flow path, and a second cavity for receiving a second part of the blower and defining the mixing chamber;
wherein the bores in the second component align with the bores in the third component to define colinear parts of the first gas flow path, second gas flow path and mixed gas flow path.

42. The device according to any one of the preceding claims, comprising a housing.

43. The device according to any one of the preceding claims, wherein the first gas flow path is an oxygen flow path.

44. A device for providing respiratory gases, the device comprising:
(a) a blower configured to generate a flow of a first gas provided through a first gases outlet;
(b) a second gas flow path configured to receive a flow of a second gas and provide the flow of the second gas through a second gases outlet;
(c) a mixing chamber configured to receive a flow of the first gas from the first gases outlet and a flow of the second gas from the second gases outlet, the received gases configured to mix in the mixing chamber to form a mixed gas, the received gases configured to travel in a mixing flow direction in the mixing chamber towards a mixed gases inlet;
wherein the mixed gas exits the mixing chamber via the mixed gases inlet providing flow to a mixed gas flow path; and
wherein the mixed gases inlet is positioned in the mixing chamber relative to one or both of the first gases outlet and second gases outlet to achieve optimal uniformity of flow within the mixed gas flow path.

45. The device according to claim 44, wherein the first gases outlet is arranged such that flow from the first gases outlet is directed in the mixing chamber away from the mixed gases inlet.

46. The device according to claim 44 or claim 45, wherein the second gases outlet is arranged such that flow from the second gases outlet is directed in the mixing chamber away from the mixed gases inlet.

47. The device according to claim 46, wherein the first gases outlet and the second gases outlet are arranged such that the first gas in the first gases outlet is directed in a first flow direction between a direction that is substantially parallel to a second flow direction of the second gas in the second gas outlet, and a direction that is substantially perpendicular to the second flow direction.

48. The device according to claim 47 wherein the first flow direction relative to the second flow direction is at an angle of about 0° to less than about 90°.

49. The device according to claim 47 or claim 48, wherein the first flow direction and the second flow direction are in a common plane.

50. The device according to any one of claims 47 to 49, wherein the mixed gases inlet is arranged such that mixed gas flow in the mixed gas flow path is directed in a mixed flow direction between a direction substantially perpendicular to one or both of the first flow direction and the second flow direction, and a direction anti-parallel to one or both of the first flow direction and the second flow direction.

51. The device according to claim 50, wherein the first direction and the second direction are in a common plane.

52. The device according to any one of claims 44 to 51, wherein the device comprises a mixed gas flow sensor for sensing flow rate of the mixed gas in the mixed gas flow path.

53. The device according to claim 52, wherein the mixed gas flow path comprises a mixed flow conditioner upstream of the mixed gas flow sensor.

54. The device according to claim 53, wherein the mixed flow conditioner is located at the mixed gases inlet.

55. The device according to claim 53 or claim 54, wherein the mixed flow conditioner has an inlet end that is shaped to be continuous with an internal wall of the mixing chamber.

56. The device according to any one of claims 53 to 55, wherein the mixed flow conditioner comprises a plurality of substantially parallel flow channels.

57. The device according to any one of claims 53 to 56, wherein the mixed flow conditioner is integral with the mixed gases inlet.

58. The device according to any one of claims 44 to 57, wherein the mixing chamber is substantially circular in cross section.

59. The device according to any one of claims 44 to 58, wherein the mixing chamber is substantially cylindrical.

60. The device according to any one of claims 44 to 59, wherein the mixing flow direction in the mixing chamber is around a central shaft.

61. The device according to claim 60, wherein the central shaft comprises a portion of the blower.

62. The device according to any one of claims 44 to 61, wherein the mixing chamber is configured such that gases travel in the mixing chamber in a spiral manner.

63. The device according to any one of claims 44 to 62, wherein the mixing chamber is comprised of a plurality of adjacent sectors, and wherein the first gases outlet and the second gases outlet are arranged in adjacent sectors of the mixing chamber.

64. The device according to claim 63, wherein the mixed gases inlet is provided in a non-adjacent sector to the first gases outlet.

65. The device according to claim 63 or claim 64, wherein the mixed gases inlet is provided in a sector that achieves optimal uniformity of flow within the mixed gases flow path.

66. The device according to any one of claims 63 to 65, wherein the plurality of sectors comprises four quadrants.

67. The device according to any one of claims 44 to 66, wherein arrangement of the second gases outlet permits flow of gases from the mixing chamber into the second gas flow path.

68. The device according to any one of claims 44 to 67, wherein one or more of the first gas, second gas and mixed gas flows comprise a flow rate of 0 L/min or greater, optionally the mixed gas flow comprises a flow rate of about 20 L/min to about 90 L/min, optionally the mixed gas flow comprises a flow rate of about 40 L/min to about 70 L/min.

69. A device for providing a flow of respiratory gases, the device comprising a pneumatic block assembly comprising a plurality of cooperating block components configured to provide, when assembled:
(b) a first through bore defining a first gas flow path;
(b) a second through bore defining a second gas flow path;
(c) a cavity defining a mixing chamber; and
(d) a third through bore defining a mixed gas flow path;
wherein the pneumatic block assembly comprises a material having an unoccupied volume comprised of the through bores and the cavities, and wherein the proportion of unoccupied volume attributable to the through bores is greater than the proportion of unoccupied volume attributable to the cavities.

70. The device according to claim 69 wherein the cavity is configured to accommodate a blower.

71. The device according to claim 69 or claim 70, wherein the unoccupied volume attributable to the through bores is more than about 50%, preferably more than about 60% and optionally about 64% of the unoccupied volume.

72. The device according to any one or claims 69 to 71, wherein the unoccupied volume attributable to the cavities is about 20% and optionally about 18% of the unoccupied volume.

73. The device according to any one of claims 69 to 72, wherein the pneumatic block assembly further comprises one or more sensor cavities, and the unoccupied volume attributable to the sensor cavities is about 20% and optionally about 18% of the unoccupied volume.

74. The device according to any one of claims 69 to 73, wherein the pneumatic block assembly comprises a metal or metal alloy into which the through bores and cavities have been machined or milled.

75. The device according to any one of claims 69 to 74, wherein the pneumatic block assembly comprises a metal or metal alloy which into which the through bores and cavities have been formed using a mould.

76. The device according to any one of claims 69 to 75, wherein the pneumatic block assembly comprises one or more thermally conductive materials.

77. The device according to any one of claims 69 to 76, wherein the pneumatic block assembly comprises one or more materials selected from a group comprising metals, metal alloys, ceramics and polymers.

78. The device according to any one of claims 69 to 77, wherein arrangement of the first gas flow path, the second gas flow path, the mixed gas flow path and the cavities accommodating the blower and defining the mixing chamber within the pneumatic block assembly provide a compact form factor.

79. The device according to any one of claims 69 to 78, wherein the pneumatic block assembly comprises a plurality of block components, and wherein a first block component provides a mounting surface to which the other block components are configured to be mounted.

80. The device according to any one of claims 69 to 79, wherein the pneumatic block assembly comprises a mounting element configured to cooperate with a mounting structure to which the device may be mounted during use.

81. The device according to any one of claims 69 to 80, wherein the device comprises a housing.

82. The device according to any one of claims 80 to 81, wherein the mounting element is provided through the housing.

83. The device according to claim 81 or claim 82, wherein the housing contains a ventilating blower configured to ventilate inside the housing.

84. The device according to claim 83, wherein the housing comprises a baffle configured to direct flow from the ventilating blower over the pneumatic block inside the housing.

85. The device according to claim 84 wherein the flow from the ventilating blower is separate from the flow of respiratory gases.

86. The device according to claim 84 or claim 85, wherein the baffle comprises one or more slots for accommodating electrical components inside the housing.

87. The device according to any one of claims 84 to 86, wherein the baffle comprises one or more structures to guide air flow from the ventilating blower to an electrical supply connector of the device.

88. The device according to any one of claims 84 to 87, wherein the baffle comprises one or more features providing structural strength mitigating one or more of sagging, compression or bending of the baffle or part thereof.

89. The device according to any one of claims 84 to 88, wherein the baffle comprises one or more features splitting air flow from the ventilating blower and optionally, guiding flows over different components of the device such as but not limited to a power distribution component of the device.

90. The device according to any one of claims 84 to 89, wherein the baffle comprises one or more hollow portions located to engage with one or more protrusions in an inside surface of the housing.

91. The device according to claim 90, wherein the one or more hollow portions comprise conical sections configured to engage with protrusions comprising screw bosses in the housing.

92. The device according to any one of claims 84 to 91, wherein the baffle comprises one or more slots configured to cooperate with a protrusion on an internal surface of the housing.

93. The device according to any one of claims 84 to 92, wherein the baffle is arranged between opposing walls of the housing.

94. A device for providing a flow of respiratory gases, the device comprising:
(a) a flow modulator having an inlet and an outlet, the flow modulator configured to provide a flow of gases through the outlet; and
(b) a flow conditioner configured to condition the flow of gases from the outlet;
wherein the flow conditioner is configured to disperse the flow of the gases entering the flow conditioner and condition the gas exiting the flow conditioner.

95. The device according to claim 94, wherein the flow conditioner comprises a first portion configured to receive and disperse the flow of gases.

96. The device according to claim 95, wherein the first portion comprises a sintered metal filter, preferably a bronze sintered filter.

97. The device according to claim 95 or claim 96, wherein the first portion comprises a cavity configured to fill with the flow of gases which is disbursed through openings in the filter when pressure within the filter exceeds a filter threshold.

98. The device according to any one of claims 94 to 97, wherein the flow conditioner comprises a second portion configured to straighten the dispersed gases.

99. The device according to claim 98, wherein the first portion comprises an external conical shape having a tip configured to be received in a corresponding recess in the second portion.

100. The device according to claim 99, wherein the recess comprises a through hole.

101. The device according to claim 99 or claim 100, wherein the tip is shaped to key with the recess in the second portion.

102. The device according to any one of claims 98 to 101, wherein the second portion comprises a plurality of openings.

103. The device according to claim 102, wherein the openings have a cross section which is substantially circular.

104. The device according to claim 102 or claim 103, wherein the openings in the second portion provide a honeycomb structure.

105. The device according to any one of claims 98 to 104, wherein the second portion comprises a plurality of parallel flow channels.

106. The device according to claim 105, wherein the plurality of flow channels have a length which may be non-uniform between the plurality of flow channels.

107. The device according to claim 105 or claim 106, wherein the plurality of flow channels are of non-uniform diameter.

108. The device according to any one of claims 105 to 107, wherein the plurality of flow channels are of non-uniform cross-sectional shape.

109. The device according to any one of claims 105 to 108, wherein the plurality of flow channels are arranged radially in the second portion.

110. The device according to any one of claims 105 to 109 wherein the plurality of flow channels are arranged in the second portion such that they are entirely within the bounds of a flow channel downstream of the flow conditioner.

111. The device according to any one of claims 94 to 110, wherein the flow modulator comprises a proportional valve.

112. The device according to any one of claims 94 to 111, wherein the flow of gases exit the outlet at a high velocity and/or a cross-sectional area of the flow of gases exiting the outlet is less than a cross-sectional area of a flow path into which it enters.

113. A device for providing a flow of respiratory gases, the device comprising:
an inlet; and
an outlet to provide the flow of respiratory gases to a patient, the outlet comprising
an outlet connector configured to couple with a delivery connector to provide the flow of respiratory gases to a patient;
wherein the outlet connector comprises an outflow end which is configured to releasably receive the delivery connector, the outflow end comprising a plurality of apertures having an opening size smaller than the delivery connector to prevent over insertion of the delivery connector into the device.

114. The device according to claim 113, wherein the plurality of apertures are positioned toward a middle portion of the outlet connector.

115. The device according to claim 113 or claim 114, wherein the outlet connector comprises an inflow end configured to receive the flow of respiratory gases into the outlet connector.

116. The device according to any one of claims 113 to 115, wherein the outflow end comprises a central opening and the plurality of apertures.

117. The device according to any one of claims 113 to 116, wherein the outlet connector is configured to provide a plurality of flow paths when coupled with the delivery connector, comprising at least a central flow path between the inflow end and the central opening, and a plurality of outer flow paths between the inflow end and the plurality of apertures.

118. The device according to claim 117, wherein the plurality of outer flow paths are substantially parallel to the central flow path.

119. The device according to any one of claims 116 to 118, wherein the central opening is configured to align with a central opening of the delivery connector.

120. The device according to any one of claims 113 to 119, wherein the outflow end comprises an internal taper configured to guide insertion of the delivery connector.

121. The device according to any one of claims 113 to 120, wherein the outflow end is configured to form a sealing engagement with the delivery connector.

122. The device according to any one of claims 113 to 121, wherein the outflow end has a smaller internal cross-section at or near the plurality of apertures or at or near a middle portion of the outlet connector compared to an internal cross-section at or near a terminal end.

123. The device according to any one of claims 113 to 122, wherein the device comprises a non-return valve between a mixed gas outlet of the device and an inflow end of the outlet connector.

124. The device according to claim 123, wherein the device comprises a pneumatic block defining the first gas flow path, the second gas flow path, the mixed gas flow path, and the mixing chamber, and wherein the non-return valve is downstream of a mixed gases outlet of the pneumatic block.

125. The device according to claim 124, wherein the outlet connector comprises a connector gasket configured to provide a substantially sealing coupling with the pneumatic block.

126. The device according to any one of claims 123 to 125, wherein the non-return valve is positioned at an angle such that gravitational forces bias the non-return valve to a closed position when the device is upright.

127. The device according to any one of claims 113 to 126, wherein the outlet connector is oriented to receive the delivery connector at an angle requiring application of a connection force having both vertical and horizontal force vectors.

128. The device according to claim 127, wherein the outlet connector is oriented at an angle of approximately 60 degrees to vertical requiring a lateral and upward connection force to be applied.
